(19) United States
(12) Patent Application Publication
(10) Pub. No.: US 2021/0113658 A1
(43) Pub. Date: Apr. 22, 2021

(54) COVERSIN FOR THE TREATMENT OF CICATRISING EYE INFLAMMATORY DISORDERS

(71) Applicant: VOLUTION IMMUNO PHARMACEUTICALS SA, 1211 Geneve 3 (CH)

(72) Inventor: Wynne H Weston-Davies, Cheltenham (GB)

(73) Assignee: VOLUTION IMMUNO PHARMACEUTICALS SA, 1211 Geneve 3 (CH)

(21) Appl. No.: 16/603,354

(22) PCT Filed: Apr. 20, 2018
(86) PCT No.: PCT/EP2018/060239

§ 371 Date: Oct. 7, 2019

(30) Foreign Application Priority Data
Apr. 21, 2017 (GB) 1706398.3
Apr. 21, 2017 (GB) 1706406.4

(51) Int. Cl. A61K 38/17; C07K 16/28; A61P 27/02

(52) U.S. Cl. CPC A61K 38/1725; A61P 27/02; C07K 16/2896

(57) ABSTRACT

The present invention provides a method and compositions for treating or preventing a cicatrising eye inflammatory disorder, in particular Sjögren's syndrome, mucous membrane pemphigoid or atopic keratoconjunctivitis. The method comprises applying, preferably topically, to the eye of a patient suffering from such a cicatrising eye inflammatory disorder a composition, preferably an optically-acceptable composition, containing a Coversin-type protein.

### CROSS REFERENCE TO RELATED APPLICATIONS

This application is a national phase entry pursuant to 35 U.S.C. § 371 of International Application No. PCT/EP2018/060239, filed Apr. 20, 2018, which claims the benefit of priority of Great Britain Application No. 1706398.3, filed Apr. 21, 2017, and Great Britain Application No. 1706406.4, filed Apr. 21, 2017, each of which is incorporated by reference herein in its entirety for any purpose.

### SEQUENCE LISTING

This application contains a Sequence Listing in computer readable form entitled “2020-09-02_01169-0016-00US_Seq_List_ST25.txt”, created Sep. 2, 2020, having a size of 32,471 bytes, which is incorporated by reference herein.

### FIELD OF THE INVENTION

The present invention relates to a method for treating or preventing cicatrising eye inflammatory disorders, such Sjögren's syndrome, mucuous membrane pemphigoid and atopic keratoconjunctivitis.

All documents mentioned in the text and listed at the end of this description are incorporated herein by reference.

### BACKGROUND TO THE INVENTION

Complement

The complement system is an essential part of the body's natural defence mechanism against foreign invasion and is also involved in the inflammatory process. More than 30 proteins in serum and at the cell surface are involved in the functioning and regulation of the complement system. Recently, it has become apparent that, as well as the approximately 35 known components of the complement system, which may be associated with both beneficial and pathological processes, the complement system itself interacts with at least 85 biological pathways with functions as diverse as angiogenesis, platelet activation, glucose metabolism and spermatogenesis.

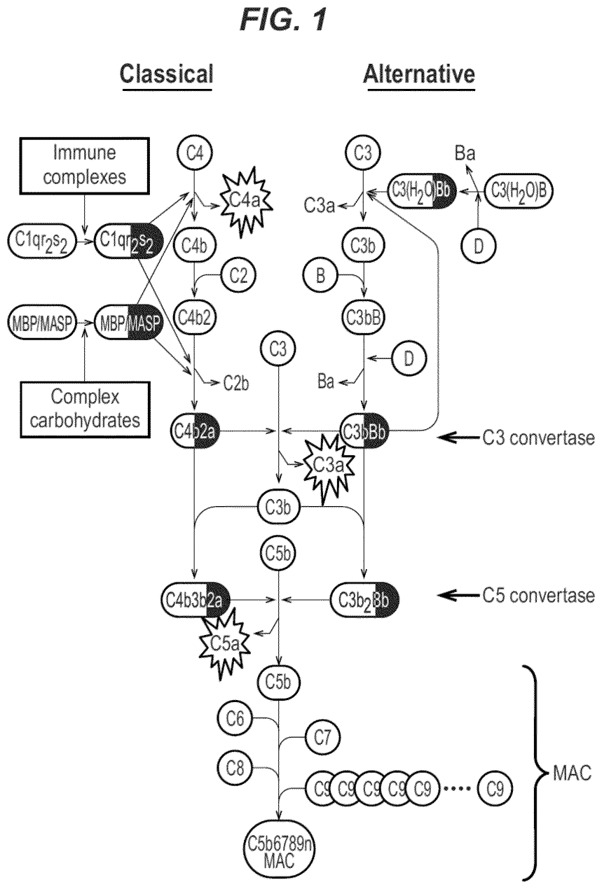

The complement system is activated by the presence of foreign antigens. Three activation pathways exist: (1) the classical pathway which is activated by IgM and IgG complexes or by recognition of carbohydrates; (2) the alternative pathway which is activated by non-self surfaces (lacking specific regulatory molecules) and by bacterial endotoxins; and (3) the lectin pathway which is activated by binding of mannan-binding lectin (MBL) to mannose residues on the surface of a pathogen. The three pathways comprise parallel cascades of events that result in complement activation through the formation of similar C3¹ and C5 convertases on cell surfaces, resulting in the release of acute mediators of inflammation (C3a and C5a) and the formation of the membrane attack complex (MAC). The parallel cascades involved in the classical and alternative pathways are shown in FIG. 1.

The classical complement pathway, the alternative complement pathway and the lectin complement pathway are herein collectively referred to as the complement pathways. C5b initiates the ‘late’ events of complement activation. These comprise a sequence of polymerization reactions in which the terminal complement components interact to form the MAC, which creates a pore in the cell membranes of some pathogens which can lead to their death. The terminal complement components include C5b (which initiates assembly of the membrane attack complex), C6, C7, C8 and C9.

Sjögren's Syndrome

Sjögren's syndrome is an autoimmune disorder. The body's immune system attacks glands that secrete fluid, such as the tear and saliva glands. The effects of Sjögren's syndrome can be widespread. Certain glands become inflamed, which reduces the production of tears and saliva, causing the main symptoms of Sjögren's syndrome, which are dry eyes and dry mouth.

In women (who are most commonly affected), the glands that keep the vagina moist can also be affected, leading to vaginal dryness.

A dry mouth can lead to other associated symptoms such as tooth decay, gum disease, a dry cough, difficulty in swallowing and chewing, a hoarse voice, difficulty in speaking, swollen salivary glands (located between the jaw and ears) and repeated fungal infections in the mouth (oral thrush), symptoms of which can include a coated or white tongue. ¹It is conventional to refer to the components of the complement pathway by the letter “C” followed by a number, such as “3”, such that “C3” refers to the third component of the complement system. Some of these components are cleaved during activation of the complement system and the cleavage products are given lower case letters after the number. Thus, C5 is cleaved into fragments which are conventionally labelled C5a and C5b. The complement proteins do not necessarily act in their number order and so the number does not necessarily give any indication of the order of action. This naming convention is used in this application.

Dry eyes can lead to burning or stinging eyes, itchy eyes, a gritty feeling in the eyes, irritated and swollen eyelids, sensitivity to light (photophobia), tired eyes and mucus discharge from the eyes. These symptoms can get worse in a windy or smoky environment, in an air-conditioned building or when travelling on an aeroplane.

In more serious cases of Sjögren's syndrome, the immune system can attack other parts of the body, causing symptoms and conditions such as dry skin, tiredness and fatigue, which are common and can lead to total exhaustion, muscle pain, joint pain, stiffness and swelling, vasculitis (inflammation of blood vessels) and difficulty in concentrating, remembering and reasoning.

Sjögren's syndrome is an autoimmune condition in that, instead of protecting the body from infection or illness, the immune system reacts abnormally and starts attacking healthy cells and tissue. In Sjögren's syndrome, the immune system attacks the tear and saliva glands and other secretory glands (the exocrine glands) throughout the body. The autoimmune reaction damages the exocrine glands so they can no longer function normally. There is some evidence that the immune system also damages the nerves that control these glands, which further reduces their effectiveness. The immune system can go on to damage other parts of the body, such as muscles, joints, blood vessels, nerves and, less commonly, organs. The reasons for this remain unknown, but research suggests that it is triggered by a combination of genetic, environmental and, possibly, hormonal factors.

Some people are thought to be more vulnerable to the syndrome when they are born and that certain events, such as an infection, can trigger the problems with the immune system.

Most researchers believe that primary Sjögren's syndrome is triggered by a combination of genetic and environmental factors. Certain people are born with specific genes which make them more vulnerable to a faulty immune system. Then, many years later, an environmental factor, possibly a common virus, triggers the immune system to stop working properly. The female hormone oestrogen also seems to play a role. Sjögren's syndrome mostly occurs in women and symptoms usually start around the time of the menopause, when oestrogen levels begin to fall. Falling oestrogen levels can contribute to dryness and this dryness could make the condition more noticeable.

Sjögren's syndrome can be associated with other autoimmune conditions, such as rheumatoid arthritis or lupus. This is known as secondary Sjögren's syndrome.

The saliva and tear glands play a vital role in protecting the mouth and eyes, which is why the symptoms of Sjögren's syndrome can be widespread and troublesome. Tears are usually only noticed on crying, but eyes are always covered by a thin layer of tears, known as a tear film. Tears are made up of a mixture of water, proteins, fats, mucus and infection-fighting cells. Tears serve several important functions. They lubricate the eye, keep the eye clean and free of dust, protect the eye against infection and help stabilise vision.

Saliva also serves several important functions including keeping the mouth and throat naturally lubricated, aiding digestion by moistening food, providing enzymes that can break down certain starches and acting as a natural disinfectant (saliva contains antibodies, enzymes and proteins that protect against some common bacterial and fungal infections).

Sjögren's syndrome can sometimes lead to complications. Sjögren's syndrome increases the risk of developing non-Hodgkin's lymphoma, which is a cancer of the lymph glands. Women with Sjögren's syndrome have an increased risk of having children with a temporary “lupus” rash or heart abnormalities. Any pregnancy must be closely monitored for potential problems. In particular, eyesight can be permanently damaged if reduced tear production is not treated. Moreover, in some instances where Sjögren's syndrome causes dry eyes, there is a cell-mediated immune reaction which causes neutrophils and other immune cells to migrate into the affected area of the eye, in particular the conjunctiva and cornea, and cause chronic fibrosing inflammation, which can damage the eye permanently.

Sjögren's syndrome most commonly affects people aged 40-60, with women accounting for about 90% of cases. Arthritis Research UK estimates that there may be up to half a million people in the UK who have Sjögren's syndrome.

Sjögren's syndrome can be difficult to diagnose because it has similar symptoms to other conditions and there is no single test for it.

There is at present no cure for Sjögren's syndrome but treatments can help control symptoms. Dry eyes and mouth can usually be helped by artificial tears and saliva. It is important to maintain good eye and mouth hygiene because the risk of developing an infection is greater. Taking care of the eyes and mouth can help prevent problems such as corneal ulcers and tooth decay. Such treatments usually only treat the symptoms of the syndrome and do not affect the underlying cause of the syndrome. At present, there is no effective topical treatment which prevents or reduces cell-mediated inflammation of the eye.

There is therefore a need to provide a method for treating or preventing cell-mediated damage to the eye caused by Sjögren's syndrome.

Cicatrising Eye Inflammatory Disorders

There are a number of other disorders which can cause chronic scarring of eye where the scarring is caused by cell-mediated damage to the eye and in particular affects the conjunctiva and cornea. Particular disorders are mucous membrane pemphigoid and atopic keratoconjunctivitis (e.g. steroid resistant atopic keratoconjunctivitis). These also include, but are not limited to, graft versus host syndrome dry eye, keratoconjunctivitis sicca, vernal keratoconjunctivitis, blepharo keratoconjunctivitis, perennial keratoconjunctivitis, ocular lupus erythematosus, ocular rosacea, trachoma, bacterial, viral or fungal keratitis, ocular herpes simplex or herpes zoster, keratoconus including, but not limited to, hereditary and traumatic keratoconus, retinitis pigmentosa, retinitis of prematurity, Down's syndrome, osteogenesis imperfecta, Addison's disease, Leber's congenital amaurosis, Ehlers-Danlos syndrome, map-dot-fingerprint corneal dystrophy, Fuch's corneal dystrophy, lattice corneal dystrophy, photokeratitis, anterior uveitis and pterygium. There are also no effective topical treatments for these disorders.

The disorders mentioned above, including Sjögren's syndrome, mucous membrane pemphigoid and atopic keratoconjunctivitis, and any other disorder in which cells, such as neutrophils and/or other immune system cells, migrate to the eye, in particular to the conjunctiva and cornea, and cause scarring damage to the eye are referred to herein as “cicatrising eye inflammatory disorders”.

Complement Inhibitors

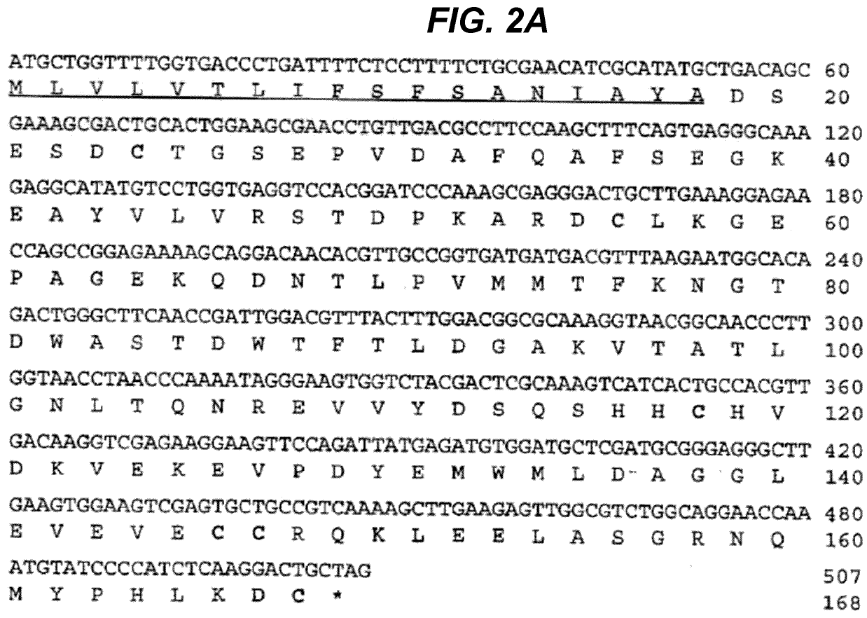

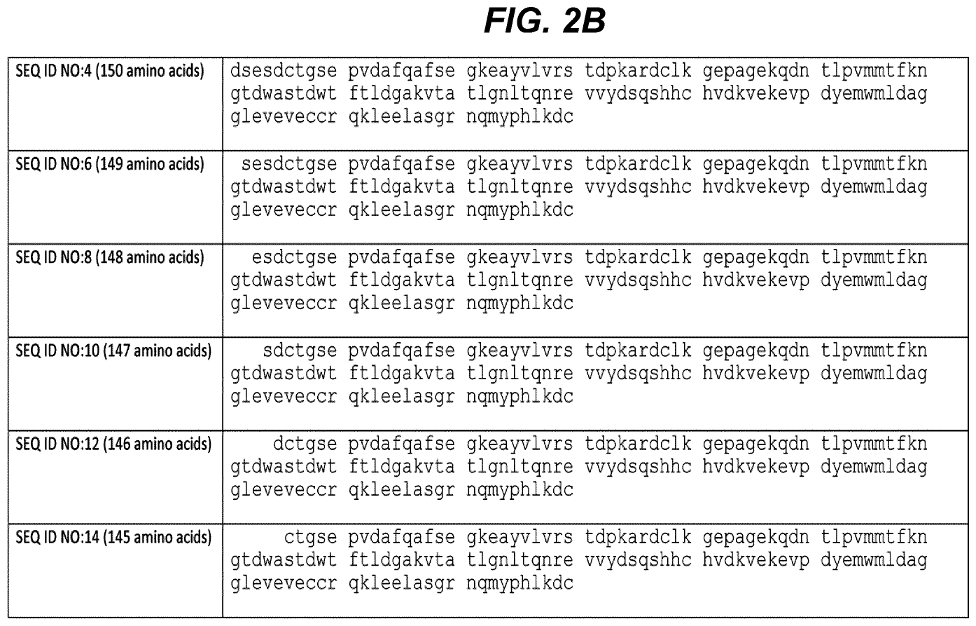

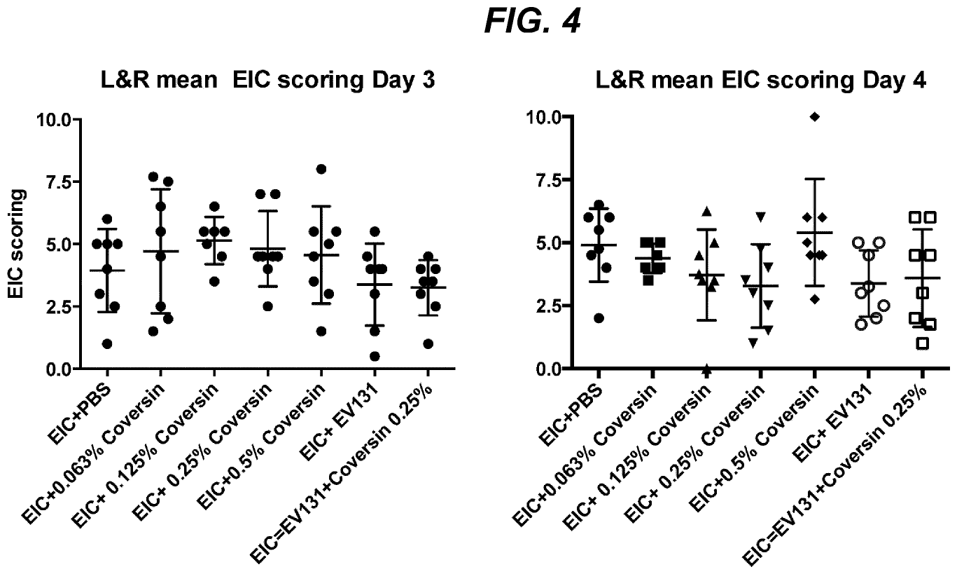

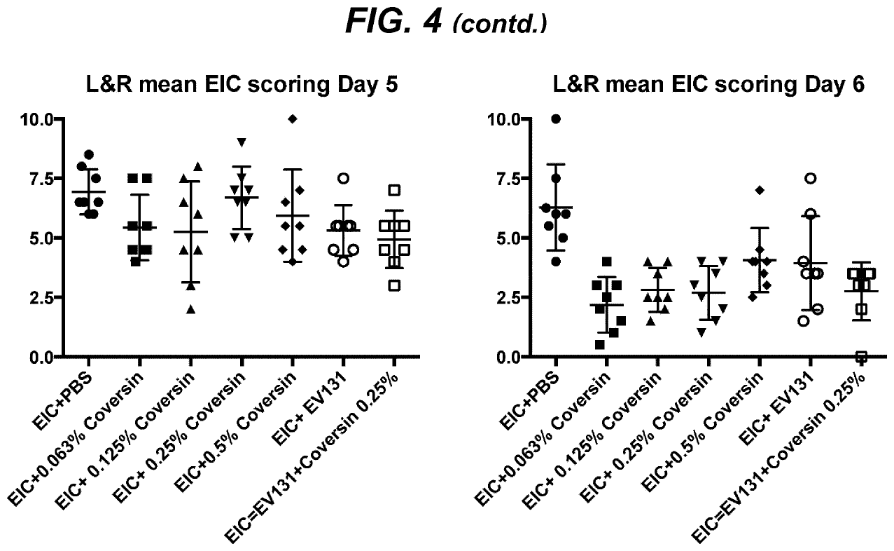

WO 2004/106369 (Evolutec Limited) relates to complement inhibitors. A particular subset of the disclosed complement inhibitors are directed at C5 and prevent C5 being cleaved into C5a and C5b by any of the complement activation pathways. A particular example of such an inhibitor of C5 cleavage is a protein produced by ticks of the species Ornithdoros moubata, which is a protein consisting of amino acids 19 to 168 of the amino acid sequence shown in FIG. 4 of WO 2004/106369. (For ease of reference, FIG. 4 of WO 2004/106369 is reproduced as FIG. 2 in this application.) In WO 2004/106369, this protein is known by the names “EV576” and “OmCI protein” and has more recently been known as “Coversin” (see, for instance, Jore et. al., Nature Structural & Molecular Biology, Structural basis for therapeutic inhibition of complement C5, published online on 28 Mar. 2016—doi:10.1038/nsmb.3196). This protein is referred to herein as “Coversin”.

In the tick, Coversin is expressed as a pre-protein having a leader sequence comprising amino acids 1 to 18 of the amino acid sequence shown in FIG. 4 of WO 2004/106369 at the N-terminal end of the mature Coversin protein. The leader sequence is cleaved off after expression.

Coversin also has the ability to inhibit leukotriene B4 (LTB-4) activity. The ability to bind LTB-4 may be demonstrated by standard in vitro assays known in the art, for example by means of a competitive ELISA between Coversin and an anti-LTB-4 antibody competing for binding to labelled LTB-4, by isothermal titration calorimetry or by fluorescence titration.

There are a number of further patent applications, such as WO 2007/028968, WO 2008/029167, WO 2008/029169, WO 2011/083317 and WO 2016/198133, which relate to the use of Coversin or functional equivalents thereof in various applications. WO 2015/185760 discloses that Coversin and its structural equivalents are effective at preventing cleavage of polymorphs of C5. There is no disclosure in any of these applications of the use of Coversin or any functional equivalent thereof in the treatment of any eye condition. All those disclosures are concerned with are conditions or diseases which are suspected of involving the activation of the complement system.

The website of Akari Therapeutics (http://akaritx.com/sjogren/) indicates that:
“Coversin has successfully completed a 60 day topical eye toxicology study, and there is evidence that Coversin has activity against eye surface inflammation. Delivery by the topical ocular route, made possible by Coversin's small molecular size, has considerable advantages both in reducing the total quantity of drug needed and, because the very small topical dose, virtually abolishes the effects of systemic complement inhibition, in reducing the risk of meningitis infection or need for prophylaxis of potential Neisseria infections.
Coversin's special physical characteristics make it an attractive candidate drug for the treatment of Sjögren's syndrome, which is not accessible by this route to antibodies like eculizumab or gene therapies, which require systemic complement inhibition. Relative to systemic diseases such as PNH and aHUS, ocular drug development is potentially rapid and new drugs typically gain marketing approval much sooner than their systemic counterparts.”

It will be seen that this disclosure only indicates that there may be activity in treating eye surface inflammation. However, there is no indication of the mechanism by which such surface inflammation is affected. The reference to Sjögren's syndrome gives no indication as to why the authors consider that Coversin will be effective against this syndrome and there is no indication that there are any data supporting this assertion. As Coversin is known to be an inhibitor of the cleavage of C5, it is assumed that the authors considered that the inflammation of the eye may be due to activation of the complement system but, again, this is unsupported by any data. It appears that the activity to which the website refers is activity against the immediate symptoms of dry eyes. There is no disclosure or suggestion that Coversin has any other effect. There is therefore still a need for a method for treating or preventing cell-mediated damage to the eye caused by Sjögren's syndrome and other cicatrising eye inflammatory disorders, in particular, mucous membrane pemphigoid or atopic keratoconjunctivitis.

### SUMMARY OF THE INVENTION

Coversin has been shown to reduce symptoms in a mouse model of cicatrising eye inflammatory disorder. The administration of Coversin leads to a reduction in the severity of symptoms or signs in the mouse model, as assessed by scoring (discussed in more detail below).

According to a first aspect of the present invention, there is provided a method for treating or preventing a cicatrising eye inflammatory disorder, in particular Sjögren's syndrome, mucous membrane pemphigoid or atopic keratoconjunctivitis, which comprises applying to a patient suffering from, or at risk of suffering from, said cicatrising eye inflammatory disorder a composition containing a protein comprising amino acids 19 to 168 of the amino acid sequence shown in FIG. 4 of WO 2004/106369 (FIG. 2 of this application, SEQ ID NO:2) or a functional equivalent thereof.

Preferably, the composition is an optically-acceptable composition and the composition is applied topically to the eye of the patient.

According to a second aspect of the present invention, there is provided a composition containing a protein comprising amino acids 19 to 168 of the amino acid sequence shown in FIG. 4 of WO 2004/106369 (FIG. 2 of this application, SEQ ID NO:2) or a functional equivalent thereof for use in a method for treating or preventing a cicatrising eye inflammatory disorder, in particular Sjögren's syndrome, mucous membrane pemphigoid or atopic keratoconjunctivitis, by applying the composition to a patient suffering from, or at risk of suffering from, said cicatrising eye inflammatory disorder.

Preferably, the composition is an optically-acceptable composition and the composition is to be applied topically to the eye of the patient.

In the following, the term “a Coversin-type protein” is used as shorthand for “a protein comprising amino acids 19 to 168 of the amino acid sequence shown in FIG. 4 of WO 2004/106369 or a functional equivalent thereof”.

The term “optically-acceptable composition” indicates a composition which can be applied to the eyes without causing damage to them. Such compositions are well known to those skilled in the art and include, for instance, artificial tears and wetting solutions used by contact lens users. Such a composition may be as simple as water, physiological saline or phosphate-buffered saline (PBS) but may also be any other buffered solution containing one or more additives. It is also known that the eyes may be treated with emulsions, ointments, creams, aerosol sprays, gels or nanoparticles for the delivery of therapeutic substances or by iontophoresis. Any of these compositions may be used in all the aspects of the present invention. Such optically-acceptable compositions are described, for instance, in Remington: The Science and Practice of Pharmacy, 22nd Edition, 2012.

The Coversin-type protein is preferably first administered to the patient no later than three days after the appearance of the symptoms of the cicatrising eye inflammatory disorder and is then administered to the patient at least once a day for up to 10 days, or even longer, after the symptoms appear. Thus, the Coversin-type protein may be administered on days 1 to 5, 1 to 6, 1 to 7, 1 to 8, 1 to 9 or 1 to 10 or longer, or days 2 to 5, 2 to 6, 2 to 7, 2 to 8, 2 to 9 or 2 to 10 or longer, or days 3 to 5, 3 to 6, 3 to 7, 3 to 8, 3 to 9 or 3 to 10 or longer, wherein day 1 is the day of the appearance of the symptoms. However, for patients suffering from genetically-based disorders, such as Sjögren's syndrome, the treatment may need to be continued for a long period, possibly for life.

Alternatively, the Coversin-type protein may be administered to the patient at any time after the appearance of the symptoms of the cicatrising eye inflammatory disorder. The treatment may continue for up to or for at least 1, 2, 3, 4, 5, 6 weeks or up to or at least 1, 2, 3, 4, 5, 6 months.

Advantageously, the Coversin-type protein is administered to the patient at least once, or at least twice a day, preferably at least three times a day.

Preferably, the topical dose of the Coversin-type protein is between 5 and 50 μg per dose, more preferably between 10 and 40 μg per dose and most preferably between 20 and 30 μg per dose. Alternatively the dose may be 50 to 200 μg per dose, e.g. 60 to 150, 70 to 125 μg per dose or approximately 125 μg per dose.

In applying a composition topically, it is usually indicated that a certain number of drops or a certain length of an ointment be applied to an eye. It will be a matter of routine to the skilled person to adjust the concentration of the Coversin-type protein in the optically-acceptable composition to ensure that the correct daily amount is administered when the application instructions are followed. For instance, one drop is usually about 40 μL and so one drop of a solution containing 0.063% w/v will contain 25.2 μg of Coversin.

Alternatively, the composition be defined in terms of its Coversin-type protein concentration. For example, the composition may comprise 0.063% w/v, 0.125% w/v or 0.25% w/v of Coversin-type protein, or 0.063% w/v to 0.25% w/v of Coversin-type protein. The composition may comprise 0.0125% w/v to 0.5% w/v of Coversin-type protein, e.g. 0.025% w/v to 0.4% w/v, 0.05% w/v to 0.3% w/v, 0.1% w/v to 0.25% w/v of Coversin-type protein

It has surprisingly been found that the effect of the Coversin-type protein in preventing or treating cell-mediated damage to the eye is seen at a relatively late stage in the treatment. It has been observed in an experimental model that the initial application of a Coversin-type protein does not have a significant effect on the symptoms but that after a delay, generally of from 1 to 7 days, more often 3 to 5 days, from first administration of the Coversin-type protein, there is a significant reduction in the symptoms and inhibition of the migration of neutrophils and other potentially inflammatory or damaging cells to the eye. Although not wishing to be bound in any way by the following, it is considered that this effect is caused by the inhibition of the cleavage of C5. However, the result is not that there is less immediate damage to the eye from the active components of the complement system, such as the MAC. Rather, the inhibition of the cleavage of C5 prevents the production of signalling compounds which otherwise would recruit neutrophils and other potentially inflammatory or damaging cells to the eye.

There is also growing evidence for the role of the pro-inflammatory mediator LTB4 in inflammation associated with granulocyte recruitment and, in particular, with eye surface inflammation (see Masoudi et al., Differences in Tear Film Biochemistry of Symptomatic and Asymptomatic Lens Wearers, Optom Vis Sci. 2017 September; 94(9):914-918; Leonardi, Allergy and allergic mediators in tears, Experimental Eye Research 117 (2013) 106-117 and Sadik et al., Neutrophils cascading their way to inflammation, Trends Immunol. 2011 October; 32(10): 452-460). Accordingly, agents for use in the present invention that inhibit the activity of LTB4 (such as Coversin, Coversin-type proteins or modified Coversin polypeptides) may have a positive effect on signs and symptoms associated with LTB4 mediated inflammation.

The subject may, as a result of the treatment, have reduced incidence of symptoms, alleviation of symptoms, inhibition or delay of occurrence or re-occurence of symptoms, or a combination thereof. Preferably the treatment gives rise to a reduction in the typical disease condition symptoms. For example, this may be manifest in reducing redness, chemosis and tearing. It may additionally or alternatively be manifest in a reduction in cell mediated damage to the eye, and/or a reduction in migration of neutrophils and/or other damaging cells to the eye.

Symptoms may be assessed according to clinical scoring using the method described by Akpek (Akpek E K, Dart J K, Watson S, Christen W, Dursun D, Yoo S, O'Brien T P, Schein O D, Gottsch J D. A randomized trial of topical cyclosporin 0.05% in topical steroid-resistant atopic keratoconjunctivitis, Ophthalmology, 2004; 111: 476-82).

A composite score of 5 symptoms and 6 signs can be used, e.g. scoring one eye only, which will be the eye judged as worst affected at each visit by the patient (or the right eye in the event that the patient judges both eyes to be equally affected). Scoring can be on a scale of 0 to 3 where 0 is unaffected, 1 is mildly affected, 2 is moderately affected and 3 is severely affected. The maximum possible score for symptoms and signs combined using this scoring system is 33.

The following symptoms can be scored:
1. Itching
2. Tearing
3. Discomfort (burning, stinging or foreign body sensation)
4. Discharge
5. Photophobia

The following signs can be scored:
1. Bulbar conjunctival hyperaemia
2. Tarsal conjunctival papillary hypertrophy
3. Punctate keratitis
4. Neovascularisation of cornea
5. Cicatrising conjunctivitis
6. Blepharitis

| | 0 | 1 | 2 | 3 |
|---|---|---|---|---|
| SYMPTOM | | | | |
| Itch | No desire | Occasional desire | Frequent need to | Constant need to rub |
| | to rub or | to rub or scratch | rub or scratch the | or scratch the eye |
| | scratch | the eye | eye | |
| | the eye | | | |
| Tearing | Normal | Positive | Intermittent, | Constant, or nearly |
| | tear | sensation of | infrequent | constant, spilling of |
| | production | fullness of the | spilling of tears | tears over the lid |
| | | conjunctival sac | over the lid | margins |
| | | without tears | margin | |
| | | spilling | | |
| | | over the lid | | |
| | | margin | | |
| Discomfort | Absent | Mild | Moderate | Severe |
| (including burning, | | | | |
| stinging, and foreign | | | | |
| body sensations) | | | | |
| Discharge | No | Small amount of | Moderate amount | Eyelids tightly matted |
| | abnormal | mucoid discharge | of mucoid | together upon |
| | discharge | noted in the lower | discharge noted | awakening, |
| | | cul-de-sac | in the lower cul- | requiring warm soaks |
| | | | de-sac and in the | to pry lids apart; warm |
| | | | marginal tear | soaks necessary to clean |
| | | | strip; | eyelids during the day |
| | | | presence of crust | |
| | | | upon awakening | |
| Photophobia | No | Mild difficulty | Moderate | Extreme photophobia, |
| | difficulty | with light causing | difficulty, | causing the patient to |
| | experienced | squinting | necessitating | stay indoors; cannot stand |
| | | | dark glasses | natural light even with |
| | | | | dark glasses |
| SIGN | | | | |
| Bulbar conjunctival | Absent | Mild | Moderate | Severe |
| hyperaemia | | | | |
| Tarsal conjunctival | No | Mild papillary | Moderate | Severe papillary |
| papillary hypertrophy | evidence | hyperaemia | papillary | hypertrophy obscuring |
| | of | | hypertrophy with | the visualization of the |
| | papillary | | oedema of the | deep tarsal vessels |
| | formation | | palpebral | |
| | | | conjunctiva and | |
| | | | hazy view of the | |
| | | | deep tarsal vessels | |
| Punctate keratitis | No | One quadrant of | Two quadrants of | Three or more |
| (superficial epithelial | evidence | punctate keratitis | punctate keratitis | quadrants of |
| keratitis and | of | | | punctate keratitis |
| punctate staining of | punctate | | | |
| the cornea with | keratitis | | | |
| fluorescein) | | | | |
| Neovascularization | No | Presence of | Presence of | Presence of |
| of cornea (new vessel | evidence | neovascularization | neovascularization | neovascularization in |
| formation, crossing | of new | in 1 quadrant of | in 2 quadrants | 3 quadrants of cornea |
| the limbus onto the | vessel | cornea | of cornea | |
| clear cornea by 2 mm) | Formation | | | |
| Cicatrizing | No | Presence of | Presence of | Symblepharon |
| conjunctivitis | evidence | subepithelial | fornix | formation |
| (superficial scarring | of | fibrosis | foreshortening | |
| of the conjunctiva) | cicatrisation | | | |
| Blepharitis | No | Presence of mild | Moderate | Severe inflammation, |
| (hyperaemia and | evidence | redness | inflammation | with cracks in the |
| oedema of eyelid | of | and oedema of | with hyperemia, | eyelid skin, loss of |
| skin with | blepharitis | the eyelid with | scales, and scurf | eyelashes, and lid |
| meibomian gland | | meibomian gland | of eyelid skin and | oedema |
| dysfunction) | | dysfunction | toothpaste | |
| | | | phenomenon | |

Preferably the treatment gives rise to a reduction in the score of one or more of the symptoms and signs shown in the table above. Preferably the treatment gives rise to a reduction in the score of any one or more (for example 2, 3, 4, 5, 6, 7, 8, 9, 10 or 11) of itching, tearing, discomfort (burning, stinging or foreign body sensation), discharge, photophobia, bulbar conjunctival hyperaemia, tarsal conjunctival papillary hypertrophy, punctate keratitis, neovascularisation of cornea, cicatrising conjunctivitis and blepharitis.

In one embodiment the treatment gives rise to a reduction in the score for itching. In one embodiment the treatment gives rise to a reduction in the score for tearing. In one embodiment the treatment gives rise to a reduction in the score for discomfort (burning, stinging or foreign body sensation). In one embodiment the treatment gives rise to a reduction in the score for discharge. In one embodiment the treatment gives rise to a reduction in the score for photophobia. In one embodiment the treatment gives rise to a reduction in the score for bulbar conjunctival hyperaemia. In one embodiment the treatment gives rise to a reduction in the score for tarsal conjunctival papillary hypertrophy. In one embodiment the treatment gives rise to a reduction in the score for punctate keratitis. In one embodiment the treatment gives rise to a reduction in the score for neovascularisation of cornea. In one embodiment the treatment gives rise to a reduction in the score for cicatrising conjunctivitis. In one embodiment the treatment gives rise to a reduction in the score for blepharitis.

A proportion of subjects will have complete resolution of symptoms and no further relapses.

In some embodiments the effects may be mediated by reduction or prevention of neutrophil involvement.

The treatment may also result in increasing the latency period before the onset of one or more stages of the disease, or between progression of disease stages. In some embodiments blistering may be prevented.

The treatment may also result in a reduction in the amount or duration of a second treatment that is required.

Thus in a further embodiment, the invention provides a method of reducing cell mediated damage to the eye in a patient with cicatrising eye inflammatory disorder, in particular Sjögren's syndrome, mucous membrane pemphigoid or atopic keratoconjunctivitis, which comprises applying to a patient suffering from, or at risk of suffering from, said cicatrising eye inflammatory disorder a composition containing a protein comprising amino acids 19 to 168 of the amino acid sequence shown in FIG. 4 of WO 2004/106369 (FIG. 2 of this application, SEQ ID NO:2) or a functional equivalent thereof.

Alternatively stated there is provided a composition containing a protein comprising amino acids 19 to 168 of the amino acid sequence shown in FIG. 4 of WO 2004/106369 (FIG. 2 of this application, SEQ ID NO:2) or a functional equivalent thereof for use in a method for reducing cell mediated damage to the eye in a patient with a cicatrising eye inflammatory disorder, in particular Sjögren's syndrome, mucous membrane pemphigoid or atopic keratoconjunctivitis, by applying the composition to a patient suffering from, or at risk of suffering from, said cicatrising eye inflammatory disorder. Where the treatment gives rise to a reduction in the amount of the second treatment, or in the duration of the treatment with the second treatment, the reduction may be up to or at least 10, 20, 30, 40, 50, 60, 70, 80% compared to the amount of the second treatment that is used in the absence of the agent of the invention. Any reference to any reduction or increase in a disease parameter is compared to said subject in the absence of the treatment. Preferably, the parameter can be quantitated and where this is the case the increase or decrease is preferably statistically significant. For example the increase or decrease may be at least 3, 5, 10, 15, 20, 30, 40, 50% compared to the parameter in the absence of treatment (e.g. before said treatment is started).

It has also been surprisingly been found that the effectiveness of the treatment is greater at lower doses than at higher doses. This is unusual in that, usually for a treatment regime, the patient is given as much of an active substance as possible without inducing unwanted side effects. It is known that Coversin generally has low side effects and so can be used at higher doses systemically. However, it has been found that the symptoms of cicatrising eye inflammatory disorders, such as Sjögren's syndrome, mucuous membrane pemphigoid and atopic keratoconjunctivitis, are reduced more using lower doses of Coversin than using higher doses. It is therefore preferred to use lower doses of the Coversin-type protein, especially in combination with use over an extended time period as referred to above. Although not wishing to be bound in any way by the following, it is considered that this effect is seen because a Coversin-type protein is generically a protein and it has been found that proteins can cause inflammation when applied topically to the eye. Therefore, using a lower dose of the Coversin-type protein may be balancing the desired inhibition of cell migration with the unwanted increase in inflammation of the eye.

The Coversin-type protein is either Coversin itself, which is a protein consisting of amino acids 19 to 168 of the amino acid sequence in FIG. 4 of WO 2004/106369 (FIG. 2 of this application, SEQ ID NO:2), or is a functional equivalent of this protein.

Coversin was isolated from the salivary glands of the tick Ornithodoros moubata. Coversin is an outlying member of the lipocalin family and is the first lipocalin family member shown to inhibit complement activation. Coversin inhibits the classical, alternative and lectin complement pathways by binding to C5 and preventing its cleavage by C5 convertase into C5a and C5b, thus inhibiting both the production of C5a, which is an active peptide, and the formation of the MAC. Coversin has been demonstrated to bind to C5 and prevent its cleavage by C5 convertase in rat, mouse and human serum with an IC₅₀ of approximately 0.02 mg/ml.

A Coversin-type protein may comprise or consist of amino acids 1 to 168 of the amino acid sequence in FIG. 4 of WO 2004/106369. The first 18 amino acids of the protein sequence given in that FIG. 4 form a signal sequence which is not required for C5 binding activity or for LTB-4 binding activity and so this may be dispensed with, for example, for efficiency of recombinant protein production (so that the mature protein is used or a protein comprising the amino acid sequence of the mature protein).

The Coversin protein has been demonstrated to bind to C5 with a Kd of 1 nM, determined using surface plasmon resonance (SPR) [Roversi, P et al Journal of Biological Chemistry 2013, 288(26) 18789-18802]. Functional equivalents of the Coversin protein preferably retain the ability to bind C5, conveniently with a Kd of less than 360 nM, more conveniently less than 300 nM, most conveniently less than 250 nM, preferably less than 200 nM, more preferably less than 150 nM, most preferably less than 100 nM, even more preferably less than 50, 40, 30, 20, or 10 nM, and advantageously less than 5 nM, wherein said Kd is determined using surface plasmon resonance, preferably in accordance with the method described in [Roversi, P et al Journal of Biological Chemistry 2013, 288(26) 18789-18802].

Coversin inhibits the classical complement pathway, the alternative complement pathway and the lectin complement pathway. Preferably, a Coversin-type protein binds to C5 in such a way as to stabilize the global conformation of C5 but not block the C5 convertase cleavage site. Binding of Coversin to C5 results in stabilization of the global conformation of C5 but does not directly block the C5 cleavage site targeted by the C5 convertases of the three activation pathways. Functional equivalents of Coversin also preferably share these properties.

Coversin has also been demonstrated to bind LTB-4. Functional equivalents of Coversin preferably also retain the ability to bind LTB-4 with a similar affinity to that of Coversin. However, this is not essential if the Coversin-type protein retains C5-binding ability and so such a Coversin-type protein does not need to bind significantly or at all to LTB-4. Coversin-type proteins which retain C5-binding ability but which do not retain LTB-4-binding activity are disclosed, for instance, in WO 2010/100396, the entire contents of which are incorporated herein by reference. Such Coversin-type proteins which have reduced or absent LTB-4-binding ability may be used in all aspects of the present invention.

Coversin has also been demonstrated to bind LTB-4. Functional equivalents of Coversin may also retain the ability to bind LTB-4 with a similar affinity to that of Coversin. If the Coversin-type protein does not retain C5-binding ability, such a Coversin-type protein should retain significant LTB-4-binding ability. Coversin-type proteins which do not retain C5-binding ability but which do retain LTB-4-binding activity are disclosed, for instance, in co-pending UK patent application No. GB 1706406.4 (Applicant's reference P070475 GB) filed on 21 Apr. 2017, as well as International application No. PCT/EP2018/XXXXXX (Applicant's reference P070475WO) filed on the same day as the present application was filed, the entire contents of which are incorporated herein by reference. Such Coversin-type proteins which have reduced or absent C5-binding activity but which retain LTB-4-binding ability may be used in all aspects of the present invention.

Such Coversin-type proteins may comprise or consist of the following sequences:

SEQ ID NO: 34 (SEQ ID NO: 5 of GB 1706406.4) is the amino acid sequence of a modified Coversin in which SEQ ID NO: 4 has been modified to change Met114 to Gln, Met116 to Gln, Leu117 to Ser, Asp118 to Asn, Ala119 to Gly, Gly120 to Ser, Gly121 to Ala, Leu122 to Asp, Glu123 to Asp and Val124 to Lys. (Coversin variant 1)

SEQ ID NO: 35 (SEQ ID NO: 6 of GB 1706406.4) is the amino acid sequence of a modified Coversin in which SEQ ID NO: 4 has been modified to change Ala44 to Asn, Met116 to Gln, Leu117 to Ser, Gly121 to Ala, Leu122 to Asp, Glu123 to Ala and Asp149 to Gly. (Coversin variant 2)

SEQ ID NO: 36 (SEQ ID NO: 7 of GB 1706406.4) is the amino acid sequence of a modified Coversin in which SEQ ID NO: 4 has been modified to change Ala44 to Asn, Met116 to Gln, Leu122 to Asp and Asp149 to Gly. (Coversin variant 3)

SEQ ID NO: 37 (SEQ ID NO: 8 of GB 1706406.4) is the amino acid sequence of a modified Coversin in which SEQ ID NO: 4 has been modified to change Ala44 to Asn. (Coversin variant 4)

SEQ ID NO: 38 (SEQ ID NO: 9 of GB 1706406.4) is the amino acid sequence of the loop between beta H and alpha2 at amino acid positions 114 to 124 of SEQ ID NO: 4 (amino acid positions 132-142 of SEQ ID NO: 2).

SEQ ID NO: 39 (SEQ ID NO: 10 of GB 1706406.4) is the amino acid sequence of the loop between beta H and alpha2 at amino acid positions 114 to 124 of SEQ ID NO: 4 in Coversin variant 1 (SEQ ID NO: 34).

SEQ ID NO: 40 (SEQ ID NO: 11 of GB 1706406.4) is the amino acid sequence of the loop between beta H and alpha2 at amino acid positions 114 to 124 of SEQ ID NO: 4 in Coversin variant 2 (SEQ ID NO: 35).

SEQ ID NO: 41 (SEQ ID NO: 12 of GB 1706406.4) is the amino acid sequence of the loop between beta H and alpha2 at amino acid positions 114 to 124 of SEQ ID NO: 4 in Coversin variant 3 (SEQ ID NO: 36).

The Coversin-type polypeptides may be described as modified Coversin polypeptides (e.g which exhibit leukotriene or hydroxyeicosanoid binding activity and reduced or absent C5 binding). References to a “modified Coversin polypeptide” are to be understood as a reference to a modified version of either SEQ ID NO: 2 or SEQ ID NO: 4 i.e. the Coversin polypeptide with or without the 18 amino acid signal sequence seen at the N-terminus of SEQ ID NO: 2.

Such polypeptides may exhibit leukotriene or hydroxyeicosanoid binding activity and reduced or absent C5 binding and can comprise SEQ ID NO: 4 in which from 1 to 30 amino acid substitutions are made, wherein

(i) in the positions 114 to 124 of SEQ ID NO: 4 one or more of the following substitutions (a)-(j) is made:

a. Met114 is replaced with Gln, Asp, Asn, Glu, Arg, Lys, Gly, Ala, Pro, His, or Thr;

b. Met116 is replaced with Gln, Asp, Asn, Glu, Arg, Lys, Gly, Ala, Pro, His, or Thr;

c. Leu117 is replaced with Ser, Asp, Asn, Glu, Arg, Lys, Gly, Ala, or Pro;

d. Asp118 is replaced with Asn, Gln, Arg, Lys, Gly, Ala, Leu, Ser, Ile, Phe, Tyr, Met Pro, His, or Thr;

e. Ala119 is replaced with Gly, Asp, Asn, Glu, Arg, Lys, Leu, Ile, Phe, Tyr, Met, Pro, or His;

f. Gly120 is replaced with Ser, Asp, Asn, Glu, Arg, Lys, Leu, Ile, Phe, Tyr, Met, Pro, or His;

g. Gly121 is replaced with Ala, Asp, Asn, Glu, Arg, Lys, Leu, Ile, Phe, Tyr, Met, Pro, or

His;

h. Leu122 is replaced with Asp, Glu, Asn, Ala, Gln, Arg, Lys, Pro, or His;

i. Glu123 is replaced with Asp, Ala, Gln, Asn, Arg, Lys, Gly, Leu, Ser, Ile, Phe, Tyr, Pro, His, or Thr;

j. Val124 is replaced with Lys, Gln, Asn, Arg, Lys, Gly, Ala, Pro, His, or Thr; or/and wherein

(ii) Ala44 in SEQ ID NO: 4 is replaced with Asn, Asp, Gln, Glu, Arg, Lys, Leu, Ile, Phe, Tyr, Met, Pro, or His;

or a fragment thereof in which up to five amino acids are deleted from the N terminus of the modified Coversin polypeptide.

LK/E binding activity as used herein refers to the ability to bind to leukotrienes and hydroxyeicosanoids including but not limited to LTB4, B4 isoleukotrienes and any hydroxylated derivative thereof, HETEs, HPETEs and EETs. LTB4 binding is of particular interest.

The modified Coversin polypeptides may consist of SEQ ID NO: 2 or 4, modified in accordance with the description below, or may comprise SEQ ID NO: 2 or 4, modified in accordance with the description below.

The unmodified Coversin polypeptide in SEQ ID NO: 2 and SEQ ID NO: 4 features a loop between beta H and alpha2 at amino acid positions 114 to 124 of SEQ ID NO: 4 (amino acid positions 132-142 of SEQ ID NO: 2). This loop has the sequence shown below:

(SEQ ID NO: 38)
-Met-Trp-Met-Leu-Asp-Ala-Gly-Gly-Leu-Glu-Val-

The first Met is at position 114 of SEQ ID NO: 4 and at position 132 of SEQ ID NO: 2.

In the modified Coversin polypeptide, the Coversin polypeptide of SEQ ID NO: 2 or SEQ ID NO: 4 is modified such that at positions 114 to 124 of SEQ ID NO: 4 one or more of the following substitutions (a)-(j) is made:

a. Met114 is replaced with Gln, Asp, Asn, Glu, Arg, Lys, Gly, Ala, Pro, His, or Thr, preferably Gln or Ala;

b. Met116 is replaced with Gln, Asp, Asn, Glu, Arg, Lys, Gly, Ala, Pro, His, or Thr, preferably Gln or Ala;

c. Leu117 is replaced with Ser, Asp, Asn, Glu, Arg, Lys, Gly, Ala, or Pro, preferably Ser or Ala;

d. Asp118 is replaced with Asn, Gln, Arg, Lys, Gly, Ala, Leu, Ser, Ile, Phe, Tyr, Met Pro, His, or Thr, preferably Asn;

e. Ala119 is replaced with Gly, Asp, Asn, Glu, Arg, Lys, Leu, Ile, Phe, Tyr, Met, Pro, or His, preferably Gly or Asn;

f. Gly120 is replaced with Ser, Asp, Asn, Glu, Arg, Lys, Leu, Ile, Phe, Tyr, Met, Pro, or His, preferably Ser or Asn;

g. Gly121 is replaced with Ala, Asp, Asn, Glu, Arg, Lys, Leu, Ile, Phe, Tyr, Met, Pro, or His, preferably Ala or Asn;

h. Leu122 is replaced with Asp, Glu, Asn, Ala, Gln, Arg, Lys, Pro, or His, preferably Asp or Ala;

i. Glu123 is replaced with Asp, Ala, Gln, Asn, Arg, Lys, Gly, Leu, Ser, Ile, Phe, Tyr, Pro, His, or Thr, preferably Asp, Ala, Gln or Asn;

j. Val124 is replaced with Lys, Gln, Asn, Arg, Lys, Gly, Ala, Pro, His, or Thr, preferably Lys or Ala.

In the modified Coversin polypeptide the Coversin polypeptide in SEQ ID NO: 2 or SEQ ID NO: 4 can be modified such that at positions 114 to 124 of SEQ ID NO: 4 one or more of the following substitutions (a)-(j) is made:

a. Met114 is replaced with Gln;

b. Met116 is replaced with Gln;

c. Leu117 is replaced with Ser;

d. Asp118 is replaced with Asn;

e. Ala119 is replaced with Gly;

f. Gly120 is replaced with Ser;

g. Gly121 is replaced with Ala;

h. Leu122 is replaced with Asp;

i. Glu123 is replaced with Asp, or Ala;

j. Val124 is replaced with Lys.

In the modified Coversin polypeptide two, three, four, five, six, seven, eight, nine, or ten of the substitutions (a)-(j) are present. Preferably two or more, five or more, or eight or more of the substitutions (a)-(j) are present.

In the modified Coversin polypeptide the Coversin polypeptide in SEQ ID NO: 2 or SEQ ID NO: 4 can be modified such that at positions 114 to 124 of SEQ ID NO: 4 the following substitutions are present:

a. Met114 is replaced with Gln;

b. Met116 is replaced with Gln;

c. Leu117 is replaced with Ser;

d. Asp118 is replaced with Asn;

e. Ala119 is replaced with Gly;

f. Gly120 is replaced with Ser;

g. Gly121 is replaced with Ala;

h. Leu122 is replaced with Asp;

i. Glu123 is replaced with Asp;

j. Val124 is replaced with Lys.

Optionally in the modified Coversin polypeptide referred to above Trp115 is not substituted. A preferred modified Coversin polypeptide has a loop between beta H and alpha2 at amino acid positions 114 to 124 of SEQ ID NO: 4 that has the sequence Gln-Trp-Gln-Ser-Asn-Gly-Ser-Ala-Asp-Asp-Lys (SEQ ID NO:39).

In the modified Coversin polypeptide, the Coversin polypeptide can be modified such that at positions 114 to 124 of SEQ ID NO: 4 the following substitutions are present:

a. Met114 is replaced with Gln, Asp, Asn, Glu, Arg, Lys, Gly, Ala, Pro, His, or Thr, preferably Gln;

b. Leu117 is replaced with Ser, Asp, Asn, Glu, Arg, Lys, Gly, Ala, or Pro, preferably Ser;

c. Gly121 is replaced with Ala, Asp, Glu, Arg, Lys, Leu, Ile, Phe, Tyr, Met, Pro, or His, preferably Ala;

d. Leu122 is replaced with Asp, Glu, Asn, Gln, Arg, Lys, Pro, or His, preferably Asp;

e. Glu123 is replaced with Asp, Ala, Gln, Asn, Arg, Lys, Gly, Leu, Ser, Ile, Phe, Tyr, Pro, His, or Thr, preferably Asp.

In more particular embodiments;

a. Met116 is replaced with Gln;

b. Leu117 is replaced with Ser;

c. Gly121 is replaced with Ala;

d. Leu122 is replaced with Asp;

e. Glu123 is replaced with Ala.

Optionally in this modified Coversin polypeptide referred to above Trp 115 is not substituted. Optionally in this embodiment Met114, Trp 115, Asp118, Ala119, Gly120 and Val124 are not substituted, or are substituted with conservative substitutions as referred to elsewhere herein. A preferred modified Coversin polypeptide has a loop between beta H and alpha2 at amino acid positions 114 to 124 of SEQ ID NO: 4 that has the sequence Met-Trp-Gln-Ser-Asp-Ala-Gly-Ala-Asp-Ala-Val (SEQ ID NO:40).

In the modified Coversin polypeptide, the Coversin polypeptide can be modified such that at positions 114 to 124 of SEQ ID NO: 4 the following substitutions are present:

a. Met116 is replaced with Gln, Asp, Asn, Glu, Arg, Lys, Gly, Ala, Pro, His, or Thr, preferably Gln;

b. Leu122 is replaced with Asp, Glu, Asn, Gln, Arg, Lys, Pro, or His, preferably Asp;

In more particular embodiments;

a. Met116 is replaced with Gln;

b. Leu122 is replaced with Asp.

Optionally in this modified Coversin polypeptide referred to above Trp 115 is not substituted. Optionally in this embodiment Met114, Trp 115, Leu117, Asp118, Ala119, Gly120, Gly121, Glu123 and Val124 are not substituted. A preferred modified Coversin polypeptide has a loop between beta H and alpha2 at amino acid positions 114 to 124 of SEQ ID NO: 4 that has the sequence Met-Trp-Gln-Leu-Asp-Ala-Gly-Gly-Asp-Glu-Val (SEQ ID NO:41).

In the modified Coversin polypeptide the Coversin polypeptide can be modified such that Ala44 in SEQ ID NO: 4 (Ala62 in SEQ ID NO: 2) is replaced with Asn, Asp, Gln, Glu, Arg, Lys, Leu, Ile, Phe, Tyr, Met, Pro, or His.

In preferred embodiments Ala44 in SEQ ID NO: 4 is replaced with Asn.

This substitution at position 44 of SEQ ID NO: 4 (or position 62 of SEQ ID NO: 2) may be made in combination with any of the other substitutions referred to herein.

In another modified Coversin polypeptide the Coversin polypeptide can be modified such that at positions 114 to 124 of SEQ ID NO: 4 one or more of the following substitutions (a)-(j) is present:

a. Met114 is replaced with Gln, Asp, Asn, Glu, Arg, Lys, Gly, Ala, Pro, His, or Thr, preferably Gln or Ala, e.g. Gln;

b. Met116 is replaced with Gln, Asp, Asn, Glu, Arg, Lys, Gly, Ala, Pro, His, or Thr, preferably Gln or Ala e.g. Gln;

c. Leu117 is replaced with Ser, Asp, Asn, Glu, Arg, Lys, Gly, Ala, or Pro, preferably Ser or Ala, e.g. Ser;

d. Asp118 is replaced with Asn, Gln, Arg, Lys, Gly, Ala, Leu, Ser, Ile, Phe, Tyr, Met Pro, His, or Thr, preferably Asn;

e. Ala119 is replaced with Gly, Asp, Glu, Arg, Lys, Leu, Ile, Phe, Tyr, Met, Pro, or His, preferably Gly or Asn, e.g. Gly;

f. Gly120 is replaced with Ser, Asp, Glu, Arg, Lys, Leu, Ile, Phe, Tyr, Met, Pro, or His, preferably Ser or Asn, e.g. Ser;

g. Gly121 is replaced with Ala, Asp, Glu, Arg, Lys, Leu, Ile, Phe, Tyr, Met, Pro, or His preferably Ala or Asn, e.g. Ala;

h. Leu122 is replaced with Asp, Glu, Asn, Gln, Arg, Lys, Pro, or His, preferably Asp or Ala, e.g. Asp;

i. Glu123 is replaced with Asp, Ala, Gln, Asn, Arg, Lys, Gly, Leu, Ser, Ile, Phe, Tyr, Pro, His, or Thr, preferably Asp, Ala, Gln or Asn, e.g. Asp or Ala;

j. Val124 is replaced with Lys, Gln, Asn, Arg, Lys, Gly, Ala, Pro, His, or Thr, preferably Lys or Ala, e.g. Lys;

and additionally Ala44 in SEQ ID NO: 4 (Ala62 in SEQ ID NO: 2) is replaced with Asn, Asp, Gln, Glu, Arg, Lys, Leu, Ile, Phe, Tyr, Met, Pro, or His, preferably Asn.

In some modified Coversin polypeptides, the Coversin polypeptide can be modified such that at positions 114 to 124 of SEQ ID NO: 4 the following substitutions are present:

a. Met116 is replaced with Gln;

b. Leu117 is replaced with Ser;

c. Gly121 is replaced with Ala;

d. Leu122 is replaced with Asp;

e. Glu123 is replaced with Ala;

and Ala44 in SEQ ID NO: 4 is replaced with Asn.

In preferred aspects of this embodiment the amino acid residues corresponding to positions 114 to 124 of SEQ ID NO: 4 are as set out in SEQ ID NO: 40.

In some modified Coversin polypeptides, the Coversin polypeptide is modified such that at positions 114 to 124 of SEQ ID NO: 4 the following substitutions are present:

a. Met116 is replaced with Gln;

b. Leu122 is replaced with Asp;

and Ala44 in SEQ ID NO: 4 is replaced with Asn

In preferred aspects of this embodiment the amino acid residues corresponding to positions 114 to 124 of SEQ ID NO: 4 are as set out in SEQ ID NO:41.

In some modified Coversin polypeptides the Coversin polypeptide can be modified such that Asp149 in SEQ ID NO: 4 is replaced with Gly, Gln, Asn, Ala, Met, Arg, Lys, Leu, Ser, Ile, Phe, Tyr, Pro, His, or Thr. In some embodiments the Coversin polypeptide is modified such that Asp149 of SEQ ID NO: 4 is replaced with Gly. This substitution at position 149 of SEQ ID NO: 4 (position 167 of SEQ ID NO: 2) may be made in combination with any of the other substitutions referred to herein.

In some modified Coversin polypeptides the Coversin polypeptide can be modified such that at positions 114 to 124 of SEQ ID NO: 4 the following substitutions are present:

a. Met116 is replaced with Gln;

b. Leu117 is replaced with Ser;

c. Gly121 is replaced with Ala;

d. Leu122 is replaced with Asp;

e. Glu123 is replaced with Ala; Ala44 in SEQ ID NO: 4 is replaced with Asn and Asp149 of SEQ ID NO: 4 is replaced with Gly149.

In preferred aspects of this embodiment the amino acid residues corresponding to positions 114 to 124 of SEQ ID NO: 4 are as set out in SEQ ID NO: 40.

In some modified Coversin polypeptides, the Coversin polypeptide can be modified such that at positions 114 to 124 of SEQ ID NO: 4 the following substitutions are present:

a. Met116 is replaced with Gln;

b. Leu122 is replaced with Asp;

Ala44 in SEQ ID NO: 4 is replaced with Asn and Asp149 of SEQ ID NO: 4 is replaced with Gly149.

In preferred aspects of this embodiment the amino acid residues corresponding to positions 114 to 124 of SEQ ID NO: 4 are as set out in SEQ ID NO: 41.

In the various aspects and embodiments of this disclosure, the modified Coversin polypeptides differ from the unmodified Coversin polypeptides in SEQ ID NO: 2 and SEQ ID NO: 4 by from 1 to 30 amino acids. Any modifications may be made to the Coversin polypeptide in SEQ ID NO: 2 and SEQ ID NO: 4 provided that the resulting modified Coversin polypeptide exhibits LK/E binding activity and reduced or absent C5 binding, compared to the unmodified Coversin polypeptide.

In some embodiments the six cysteine amino acids at positions 6, 38, 100, 128, 129, 150 of SEQ ID NO: 4 are retained in the modified Coversin polypeptides of the invention.

In some modified Coversin polypeptides, Asn60 and Asn84 in SEQ ID NO: 4 are each replaced with Gln. This modification can be carried out by site directed mutagenesis to prevent N-linked hyperglycosylation when the polypeptide is expressed in yeast.

In some modified Coversin polypeptides one or more of the following amino acids in SEQ ID NO: 4 are thought to be involved in binding to LTB4 and may therefore be retained in unmodified form: Phe18, Tyr25, Arg36, Leu39, Gly41, Pro43, Leu52, Val54, Met56, Phe58, Thr67, Trp69, Phe71, Gln87, Arg89, His99, His101, Asp103, and Trp115. In some modified Coversin polypeptides, at least five, ten or fifteen, or all of these amino acids are retained in unmodified form in the modified Coversin polypeptides of the invention. In some modified Coversin polypeptides one or more of these amino acids may be conservatively substituted.

In some modified Coversin polypeptides up to five, ten or fifteen, or all of these amino acids are conservatively substituted in the modified Coversin polypeptides of the invention.

Amino acids at the following positions in SEQ ID NO: 4 are highly conserved between Coversin and TSGP2 and TSGP3: 5, 6, 11, 13-15, 20-21, 24-27, 29-32, 35-41, 45, 47-48, 50, 52-60, 64, 66, 69-81, 83, 84, 86, 90-94, 97-104, 112-113, 115, 125-129, 132-139, 145, 148, and 150.

Amino acids at the following positions in SEQ ID NO: 4 are thought to be involved in binding to LTB4 and/or are highly conserved between Coversin and TSGP2 and TSGP3: 5, 6, 11, 13-15, 18, 20-21, 24-27, 29-32, 35-41, 43, 45, 47-48, 50, 52-60, 64, 66, 67, 69-81, 83, 84, 86, 87, 89, 90-94, 97-104, 112-113, 115, 125-129, 132-139, 145, 148, and 150.

Amino acids at the following positions in SEQ ID NO: 4 are thought to be involved in binding to LTB4 and/or are highly conserved between Coversin and TSGP2 and TSGP3: 5, 6, 11, 13-15, 18, 20-21, 24-25, 27, 30-32, 35-41, 43, 47-48, 50, 52-60, 64, 66, 67, 69-81, 83, 84, 86, 87, 89, 90-94, 98, 100, 102-104, 112-113, 115, 126, 128-129, 132-139, 145, 148, and 150.

In some modified Coversin polypeptides therefore the above amino acids are retained in unmodified form. In some embodiments, at least five, ten or fifteen, or all of these amino acids are retained in unmodified form in the modified Coversin polypeptides of the invention. In some embodiments one or more of these amino acids may be conservatively substituted. In some embodiments up to five, ten or fifteen, twenty, twenty five, 30, 40, 50 or all of these amino acids are conservatively substituted in the modified Coversin polypeptides of the invention

The modified Coversin polypeptides referred to herein typically differ from SEQ ID NO: 2 or SEQ ID NO: 4 by from 1 to 30, preferably from 2 to 25, more preferably from 3 to 20, even more preferably from 4 to 15 amino acids. Typically the difference will be 5 to 12, or 6 to 10 amino acid changes. For example, from 1 to 30, or 2 to 25, 3 to 30, 4 to 15, 5 to 12, or 6 to 10 amino acid substitutions may be made in SEQ ID NO: 2 or SEQ ID NO: 4.

Modified Coversin polypeptides which have the loop between beta H and alpha2 at amino acid positions 114 to 124 of SEQ ID NO: 4 (amino acid positions 132-142 of SEQ ID NO: 2) as set out in SEQ ID NO: 39 have 10 amino acid substitutions compared to SEQ ID NO: 4 as a result of the presence of this loop. In some embodiments, the modified Coversin polypeptides referred to herein preferably therefore have 1-15, 2-10, 3-5, or up to 2, 3, 4 or 5 additional substitutions compared to SEQ ID NO: 4 beyond those that are set out in SEQ ID NO: 34 (e.g. in the loop of SEQ ID NO: 39).

Modified Coversin polypeptides which have the loop between beta H and alpha2 at amino acid positions 114 to 124 of SEQ ID NO: 4 (amino acid positions 132-142 of SEQ ID NO: 2) as set out in SEQ ID NO: 40 have 5 amino acid substitutions compared to SEQ ID NO: 4 as a result of the presence of this loop. In some embodiments, the modified Coversin polypeptides referred to herein preferably therefore have 1-20, 2-15, 3-10, or up to 2, 3, 4, 5, 6, 7, 8, 9, 10 additional substitutions compared to SEQ ID NO: 4 beyond those that are set out in SEQ ID NO: 35 (e.g. in the loop of SEQ ID NO: 40). The additional substitutions preferably include substitutions at position 44 and 149, as set out elsewhere herein.

Modified Coversin polypeptides which have the loop between beta H and alpha2 at amino acid positions 114 to 124 of SEQ ID NO: 4 (amino acid positions 132-142 of SEQ ID NO: 2) as set out in SEQ ID NO: 41 have 2 amino acid substitutions compared to SEQ ID NO: 4 as a result of the presence of this loop. In some embodiments, the modified Coversin polypeptides preferably therefore have 1-25, 2-12, 3-15, or up to 2, 3, 4, 5, 6, 7, 8, 9, 10, 11, 12, 13, 14, 15 additional substitutions compared to SEQ ID NO: 4 beyond those that are set out in SEQ ID NO: 36 (e.g. substitutions in the loop of SEQ ID NO: 41). The additional substitutions preferably include substitutions at position 44 and 149, as set out elsewhere herein.

Modified Coversin polypeptides which have the substitution at position 44 of SEQ ID NO: 4 as set out elsewhere herein preferably have 1-25, 2-12, 3-15, or up to 2, 3, 4, 5, 6, 7, 8, 9, 10, 11, 12, 13, 14, 15 additional substitutions compared to SEQ ID NO: 4.

Substitutions other than those explicitly referred to above are preferably conservative substitutions, for example according to the following Table. Amino acids in the same block in the second column and preferably in the same line in the third column may be substituted for each other:

| | | |
|---|---|---|
| Aliphatic | Non-polar | G A P |
| | | I L V |
| | Polar - uncharged | C S T M |
| | | N Q |
| | Polar - charged | D E |
| | | K R |
| Aromatic | | H F W Y |

Preferred modified Coversin polypeptides comprise or consist of the amino acid sequences set out in one of SEQ ID NO:34, 35, 36, 37.

The present invention also encompasses use of fragments of the modified Coversin polypeptide referred to above in which up to five amino acids are deleted from the N terminus of the modified Coversin polypeptide. The fragment may correspond to 1, 2, 3, 4 or 5 deletions from the N terminus of the modified Coversin polypeptide. Deletions from other positions in the amino acid sequence of SEQ ID NO: 2 or SEQ ID NO: 4 are also envisaged as forming part of the invention, if the resulting polypeptide retains the LK/E binding activity of the modified Coversin and has reduced or absent complement inhibitor activity.

It has been demonstrated that RaCI proteins also bind to C5 and can inhibit conversion of C5 to C5a and C5b by such binding. RaCI proteins are described in the Jore paper cited above, the entire contents of which are incorporated herein by reference. Such proteins are described in more detail in WO 2015/185945, the entire contents of which are incorporated herein by reference.

As set forth in WO 2015/185945, in a first aspect, the invention of WO 2015/185945 provides an isolated polypeptide comprising or consisting of:
(a) the amino acid sequence of any one of SEQ ID Nos: 1, 2, 3, 4, 5, 6, 7, 8, 9, 10, 11 or 12;
(b) a variant amino acid sequence having at least 60% sequence identity to (a);
(c) an amino acid sequence having at least 70%, 75%, 80%, 90%, 95%, 98% or 99% sequence identity to (a); or
(d) an active fragment of (a), (b) or (c) that is at least 40, 50, 60, 65, 70 or 75 amino acids in length,

wherein the sequence identity numbers and the determination of sequence identity are as disclosed in WO 2015/185945. Any of these polypeptides maybe used in all aspects of the present invention.

For ease of reference, sequences referred to in WO2015/185945 are given sequence numbers in the present application, as set out below:

| SEQ ID NO in | SEQ ID NO in |
|---|---|
| WO2015/185945 | present application |
| 1 | 22 |
| 2 | 23 |
| 3 | 24 |
| 4 | 25 |
| 5 | 26 |
| 6 | 27 |
| 7 | 28 |
| 8 | 29 |
| 9 | 30 |
| 10 | 31 |
| 11 | 32 |
| 12 | 33 |

Monoclonal antibodies and small molecules which bind to and inhibit cleavage of C5 have been developed and are in development to treat various diseases (Ricklin D & Lambris J, Nature Biotechnology, 25:1265-1275 (2007)), in particular PNH, psoriasis, rheumatoid arthritis, systemic lupus erythematosus and transplant rejection. Any of these monoclonal antibodies and small molecules may also be used in all aspects of the present invention. However, some of these monoclonal antibodies do not bind to C5 from subjects with certain C5 polymorphisms and are thus ineffective in these subjects (Nishimura, J et al., New Engl J. Med., 30; 7: 632-639 (2014)). Preferably, the Coversin-type protein is not an antibody but is a protein which binds to and inhibits cleavage of not only wild-type C5 but also C5 from subjects with C5 polymorphisms (e.g. C5 polymorphisms that render treatment by eculizumab ineffective, or reduce the efficacy of treatment with eculizumab). The term “C5 polymorphism” includes any version of C5 which has been changed by insertion, deletion, amino acid substitution, a frame-shift, truncation, any of which may be single or multiple, or a combination of one or more of these changes compared to the wild-type C5. In a human subject, the wild-type C5 is the C5 protein with accession number NP_001726.2; version GI:38016947. Examples of C5 polymorphisms include polymorphisms at position 885, e.g. Arg885Cys (encoded by c.2653C>T), p.Arg885His (encoded by c.2654G>A) and Arg885Ser which decrease the effectiveness of the monoclonal antibody eculizumab [Nishimura, J et al., New Engl J. Med., 30; 7: 632-639 (2014).

The ability of a Coversin-type protein to bind C5, including C5 from subjects with C5 polymorphisms, may be determined by standard in vitro assays known in the art, for example by surface plasmon resonance or by Western blotting following incubation of the protein on the gel with labelled C5.

The ability of an agent to bind C5, including C5 from subjects with C5 polymorphisms, e.g. C5 polymorphisms that render treatment by eculizumab ineffective, or reduce the efficacy of treatment with eculizumab may be determined by standard in vitro assays known in the art, for example by surface plasmon resonance or western blotting following incubation of the protein on the gel with labelled C5. Preferably, the Coversin-type protein binds C5, either wild-type and/or C5 from subjects with C5 polymorphisms, e.g. C5 polymorphisms that render treatment by eculizumab ineffective, or reduce the efficacy of treatment with eculizumab, with a Kd of less than 360 nM, more conveniently less than 300 nM, most conveniently less than 250 nM, preferably less than 200 nM, more preferably less than 150 nM, most preferably less than 100 nM, even more preferably less than 50, 40, 30, 20, or 10 nM, and advantageously less than 5 nM, wherein said Kd is determined using surface plasmon resonance, preferably in accordance with the method described in [Roversi et al, supra].

It may show higher, lower or the same affinity for wild-type C5 and C5 from subjects with C5 polymorphisms, e.g. C5 polymorphisms that render treatment by eculizumab ineffective, or reduce the efficacy of treatment with eculizumab.

The ability of a Coversin-type protein to inhibit complement activation may be determined by measuring its ability to inhibit complement activation in serum. For example, complement activity in the serum can be measured by any means known in the art or described herein.

Where modified Coversin polypeptides are used that have reduced or absent C5-binding activity but which retain LTB-4-binding ability, C5 binding may for example, reduced by at least 2, 5, 10, 15, 20, 50, 100 fold, or eliminated relative to the binding exhibited by the unmodified Coversin polypeptide in SEQ ID NO: 2 or 4. C5 binding may e.g. be reduced by at least 50%, 60%, 70%, 80%, 90% or 95% relative to the unmodified Coversin polypeptide in SEQ ID NO: 2 or 4. The modified Coversin polypeptides may bind C5 with a KD greater than 1 micromolar as determined by Surface Plasma Resonance according to the method described in Roversi et al. (2013) J Biol Chem. 288, 18789-18802, or as set out in Example 2 of GB1706406.4.

The Coversin-type protein may also have the function of inhibiting eicosanoid activity.

The Coversin-type protein may inhibit LTB-4 activity, but this is not necessary. In particular, the Coversin-type protein may bind to LTB-4. The ability of a Coversin-type protein to bind to LTB-4 may be determined by standard in vitro assays known in the art, for example by means of a competitive ELISA between Coversin and an anti-LTB-4 antibody competing for binding to labelled LTB-4, by isothermal titration calorimetry or by fluorescence titration.

Data obtained using fluorescence titration shows that Coversin binds to LTB4 with a Kd of between 200 and 300 pM. For example, Binding activity for LTB4 (Caymen Chemicals, Ann Arbor, Mich., USA) in phosphate buffered saline (PBS) can be quantified in a spectrofluorimeter e.g. a LS 50 B spectrofluorimeter (Perkin-Elmer, Norwalk, Conn., USA). This may be carried out by may be carried out as follows:

Purified 100 nM solutions of Coversin, in 2 mL PBS were applied in a quartz cuvette (10 mm path length; Hellma, Mühlheim, Germany) equipped with a magnetic stirrer. Temperature was adjusted to 20° C. and, after equilibrium was reached, protein Tyr/Trp fluorescence was excited at 280 nm (slit width: 15 nm). The fluorescence emission was measured at 340 nm (slit width: 16 nm) corresponding to the emission maximum. A ligand solution of 30 μM LTB4 in PBS was added step-wise, up to a maximal volume of 20 μL (1% of the whole sample volume), and after 30 s incubation steady state fluorescence was measured. For calculation of the KD value, data was normalized to an initial fluorescence intensity of 100%, the inner filter effect was corrected using a titration of 3 μM N-acetyl-tryptophanamide solution and data was plotted against the corresponding ligand concentration. Then, non-linear least squares regression based on the law of mass action for bimolecular complex formation was used to fit the data with Origin software version 8.5 (OriginLab, Northampton, Mass., USA) using a published formula (Breustedt et al., 2006: Comparative ligand-binding analysis of ten human lipocalins. Biochim Biophys Acta 1764(2):161-173.).

Coversin may bind LTB4 with an with a Kd of less than 1 nM, more conveniently less than 0.9 nM, most conveniently less than 0.8 nM, preferably less than 0.7 nM, more preferably less than 0.6 nM, most preferably less than 0.5 nM, even more preferably less than 0.4 nM, and advantageously less than 0.3 nM, wherein said Kd is determined using fluorescence titration, preferably in accordance with the method above. The Coversin-type protein preferably shares these properties. In certain embodiments, the Coversin-type protein binds to both wild-type C5 and C5 from subjects with C5 polymorphisms e.g. C5 polymorphisms that render treatment by eculizumab ineffective, or reduce the efficacy of treatment with eculizumab and to LTB-4. In other embodiments, the Coversin-type protein is a modified Coversin polypeptide as described above, that has reduced or no C5 binding, but which binds to LTB4, e.g. as described herein.

The Coversin-type proteins (e.g. the modified Coversin polypeptides which have reduced or absent C5-binding activity but which retain LTB-4-binding ability) may e.g. bind LTB4 with an with a Kd of less than 5 nM, 2 nM or 1 nM, more conveniently less than 0.9 nM, most conveniently less than 0.8 nM, preferably less than 0.7 nM, more preferably less than 0.6 nM, most preferably less than 0.5 nM, even more preferably less than 0.4 nM, and advantageously less than 0.3 nM, wherein said Kd is determined using fluorescence titration, preferably in accordance with the method above.

The Coversin-type protein may thus act to prevent the cleavage of C5 by C5 convertase into complement C5a and complement C5b and also to inhibit LTB-4 activity, or it may be a a modified Coversin polypeptide as described above, that has reduced or no C5 binding, but which binds to LTB4, e.g. as described herein.

Using a Coversin-type protein which binds to both C5 and LTB-4 is particularly advantageous. On the basis of the data presented below, the inventors consider, without being bound to this consideration, that C5 and the eicosanoid pathway both contribute to the observed pathology in cicatrising eye inflammatory disorders, in particular Sjögren's syndrome, mucuous membrane pemphigoid and atopic keratoconjunctivitis. Thus, by using a single Coversin-type protein which inhibits multiple pathways involved in the inflammatory effects of complement-mediated diseases and disorders, an enhanced effect can be achieved, compared to using an agent which inhibits only a single pathway involved in the inflammatory effects of complement-mediated diseases and disorders. There are furthermore practical advantages associated with administering a single molecule.

Preferably, the Coversin-type protein is derived from a haematophagous arthropod. The term “haematophagous arthropod” includes all arthropods that take a blood meal from a suitable host, such as insects, ticks, lice, fleas and mites. Preferably, the Coversin-type protein is derived from a tick, preferably from the tick Ornithodoros moubata.

A functional equivalent of Coversin may be a homologue or fragment of Coversin which retains its ability to bind to C5, either wild-type C5 or C5 from a subject with a C5 polymorphism e.g. C5 polymorphisms that render treatment by eculizumab ineffective, or reduce the efficacy of treatment with eculizumab, and to prevent the cleavage of C5 by C5 convertase into C5a and C5b. The homologue or fragment may also retain its ability to bind LTB-4. It may retain its ability to bind LTB-4 but not its ability to bind to C5.

A functional equivalent of Coversin may also be a molecule which is structurally similar to Coversin or which contains similar or identical tertiary structure, particularly in the environment of the active site or active sites of Coversin which binds to C5, either wild-type C5 or C5 from a subject with a C5 polymorphism, and/or LTB-4, such as synthetic molecules. The precise amino acid residues in Coversin which are required for binding to C5 and to LTB-4 are set out on the Jore et. al. reference given above.

Homologues include paralogues and orthologues of Coversin as explicitly identified in FIG. 4 of WO 2004/106369, including, for example, the Coversin protein from other tick species, including Rhipicephalus appendiculatus, R. sanguineus, R. bursa, A. americanum, A. cajennense, A. hebraeum, Boophilus microplus, B. annulatus, B. decoloratus, Dermacentor reticulatus, D. andersoni, D. marginatus, D. variabilis, Haemaphysalis inermis, Ha. leachii, Ha. punctata, Hyalomma anatolicum anatolicum, Hy. dromedarii, Hy. marginatum marginatum, Ixodes ricinus, I. persulcatus, I. scapularis, I. hexagonus, Argas persicus, A. reflexus, Ornithodoros erraticus, O. moubata moubata, O. m. porcinus, and O. savignyi.

Homologues also include proteins from mosquito species, including those of the Culex, Anopheles and Aedes genera, particularly Culex quinquefasciatus, Aedes aegypti and Anopheles gambiae; flea species, such as Ctenocephalides fells (the cat flea), horseflies, sandflies, blackflies, tsetse flies, lice, mites, leeches and flatworms, which have equivalent function to Coversin. Homologues also include the three other forms of Coversin, of around 18 kDa, which exist in O. moubata.

Methods for the identification of homologues of Coversin will be clear to those of skill in the art. For example, homologues may be identified by homology searching of sequence databases, both public and private. Conveniently, publicly-available databases are used, although private or commercially-available databases will be equally useful, particularly if they contain data not represented in the public databases. Primary databases are sites where primary nucleotide or amino acid sequence data are deposited and may be publicly or commercially available. Examples of publicly-available primary databases include the GenBank database (http://www.ncbi.nlm.nih.gov/), the EMBL database (http://www.ebi.ac.uk/), the DDBJ database (http://www.ddbj.nig.ac.jp/), the SWISS-PROT protein database (http://expasy.hcuge.ch/), the PIR database (http://pir.georgetown.edu/), the TrEMBL database (http://www.ebi.ac.uk/), the TIGR databases (see http://www.tigr.org/tdb/index.html), the NRL-3D database (http://www.nbrfa.georgetown.edu), the Protein Data Base (http://www.rcsb.org/pdb), the NRDB database (ftp://ncbi.nlm.nih.gov/pub/nrdb/README), and the OWL database (http://www.biochem.ucl.ac.uk/bsm/dbbrowser/OWL/). Examples of publicly-available secondary databases are the PROSITE database (http://expasy.hcuge.ch/sprot/prosite.html), the PRINTS database (http://iupab.leeds.ac.uk/bmb5dp/prints.html), the Profiles database (http://ulrec3.unil.ch/software/PFSCAN_form.html), the Pfam database (http://www.sanger.ac.uk/software/pfam), the Identify database (http://dna.stanford.edu/identify/) and the Blocks database (http://www.blocks.fhcrc.org).

Examples of commercially-available databases or private databases include PathoGenome (Genome Therapeutics Inc.) and PathoSeq (previously of Incyte Pharmaceuticals Inc.).

Typically, greater than 30% identity between two polypeptides (preferably over a specified region, such as the active site) is considered to be an indication of functional equivalence and thus an indication that two proteins are homologous. Preferably, proteins that are homologues have a degree of sequence identity with Coversin of greater than 60%. More preferred homologues have degrees of identity of greater than 70%, 80%, 90%, 95%, 98% or 99%, with Coversin. Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1]. The % identity may be over the full length of the relevant reference sequence (e.g. amino acids 19-168 of FIG. 4 of WO 2004/106369 (FIG. 2 of this application, SEQ ID NO:2) or amino acids 1-168 of FIG. 4 of WO 2004/106369 (FIG. 2 of this application, SEQ ID NO:2).

Coversin-type proteins thus can be described by reference to a certain % amino acid sequence identity to a reference sequence e.g. amino acids 19-168 of FIG. 4 of WO 2004/106369 (FIG. 2 of this application, SEQ ID NO:2) or amino acids 1-168 of FIG. 4 of WO 2004/106369 (FIG. 2 of this application, SEQ ID NO:2) e.g. as a protein comprising or consisting of a sequence having at least 60%, 70%, 80%, 90%, 95%, 98% or 99% identity to amino acids 19-168 of FIG. 4 of WO 2004/106369 (FIG. 2 of this application, SEQ ID NO:2) or amino acids 1-168 of FIG. 4 of WO 2004/106369 (FIG. 2 of this application, SEQ ID NO:2), Where the Coversin-type protein comprises said sequence, the Coversin-type protein may be a fusion protein (with e.g. another protein, e,g. a heterologous protein). Suitable second proteins are discussed below.

Some functional equivalents of Coversin are shown in FIG. 2a and include mutants containing amino acid substitutions, insertions or deletions compared to the wild-type sequence, for example, of 1, 2, 3, 4, 5, 7, 10 or more amino acids (e.g. deletions from the N or C terminus) provided that such mutants retain the ability to bind wild-type C5 and/or C5 from subjects with a C5 polymorphism (e.g. C5 polymorphisms that render treatment by eculizumab ineffective, or reduce the efficacy of treatment with eculizumab) and/or LTB-4. This is relative to the relevant reference sequence (i.e. amino acids 19-168 of FIG. 4 of WO 2004/106369 (FIG. 2 of this application) or amino acids 1-168 of said FIG. 4 (FIG. 2 of this application)). Mutants may include proteins containing conservative amino acid substitutions which do not affect the function or activity of the protein in an adverse manner. Functional equivalents of Coversin also include natural biological variants (e.g. allelic variants or geographical variations within the species from which Coversin is derived). Mutants with improved ability to bind wild-type C5 and/or C5 from subjects with a C5 polymorphism (e.g. C5 polymorphisms that render treatment by eculizumab ineffective, or reduce the efficacy of treatment with eculizumab) and/or LTB-4 may also be designed through systematic or directed mutation of specific residues in the Coversin sequence.

Functional equivalents of Coversin include fragments and homologues of the Coversin protein provided that such fragments retain the ability to bind wild-type C5 and/or C5 from subjects with a C5 polymorphism (e.g. C5 polymorphisms that render treatment by eculizumab ineffective, or reduce the efficacy of treatment with eculizumab) and/or LTB-4. Fragments may include, for example, polypeptides derived from Coversin or a homologue of Coversin which are less than 150 amino acids, less than 145, 140, 135, 130, 125, 100, 75, 50 or even 25 amino acids or less, provided that these fragments retain the ability to bind to wild-type C5 and/or C5 from subjects with a C5 polymorphism (e.g. C5 polymorphisms that render treatment by eculizumab ineffective, or reduce the efficacy of treatment with eculizumab) and/or LTB-4. Fragments may include, for example, polypeptides derived from the Coversin protein sequence (or homologue) which are at least 150 amino acids, at least 145, amino acids, provided that these fragments retain the ability to bind to complement wild-type C5 and/or C5 from subjects with a C5 polymorphism (e.g. C5 polymorphisms that render treatment by eculizumab ineffective, or reduce the efficacy of treatment with eculizumab) and/or LTB4.

Any functional equivalent of Coversin preferably retains the pattern of cysteine residues that is found in Coversin. For example, said functional equivalent preferably comprises six cysteine residues that are spaced relative to each other at a distance of 32 amino acids apart, 62 amino acids apart, 28 amino acids apart, 1 amino acid apart and 21 amino acids apart as arranged from the amino terminus to the carboxyl terminus of the sequence shown in FIG. 4 of WO 2004/106369 (FIG. 2 of this application, SEQ ID NO:2). Exemplary fragments of Coversin protein are disclosed in SEQ ID NO: 4, SEQ ID NO: 6, SEQ ID NO: 8, SEQ ID NO: 10, SEQ ID NO: 12, SEQ ID NO: 14. The DNA encoding the corresponding fragments are disclosed in SEQ ID NO: 3, SEQ ID NO: 5, SEQ ID NO: 7, SEQ ID NO: 9, SEQ ID NO: 11, SEQ ID NO: 13.

Functional equivalents of Coversin include not only fragments of the O. moubata Coversin that is explicitly identified herein in FIG. 4 of WO 2004/106369 (FIG. 2 of this application, SEQ ID NO:2), but also fragments of homologues of this protein, as described above. Such fragments of homologues will typically possess greater than 60% identity with fragments of Coversin, although more preferred fragments of homologues will display degrees of identity of greater than 70%, 80%, 90%, 95%, 98% or 99% with fragments of Coversin of FIG. 4 of WO 2004/106369 (FIG. 2 of this application, SEQ ID NO:2). Preferably, such fragment will retain the cysteine spacing referred to above. Fragments with improved properties may, of course, be rationally designed by systematic mutation or fragmentation of the wild-type sequence followed by appropriate activity assays. Fragments may exhibit similar or greater affinity for C5, either the wild-type or a polymorphic variant of C5 or both, and/or LTB-4 as Coversin. These fragments may be of a size described above for fragments of Coversin.

A functional equivalent may also be a fusion protein, obtained, for example, by cloning a polynucleotide encoding Coversin or a functional equivalent thereof in frame to the coding sequences for a heterologous protein sequence. The term “heterologous”, when used herein, is intended to designate any polypeptide other than Coversin protein or its functional equivalent. Example of heterologous sequences that can be comprised in a soluble fusion protein either at N- or at C-terminus are the following: extracellular domains of membrane-bound protein, immunoglobulin constant regions (Fc regions), PAS or XTEN or similar unstructured polypeptides, multimerization domains, domains of extracellular proteins, signal sequences, export sequences or sequences allowing purification by affinity chromatography. Many of these heterologous sequences are commercially available in expression plasmids since these sequences are commonly included in fusion proteins in order to provide additional properties without significantly impairing the specific biological activity of the protein fused to them (Terpe K, Appl Microbiol Biotechnol, 60: 523-33, 2003). Examples of such additional properties are a longer lasting half-life in body fluids, tissue targeting, extracellular localization or easier purification as allowed by a tag such as a histidine, GST, FLAG, avidin or HA tag. Fusion proteins may additionally contain linker sequences (e.g. 1-50 amino acids in length), such that the components are separated by this linker. However, it is preferred not to use fusion proteins in any of the aspects of the invention.

Fusion proteins are thus examples of proteins comprising a Coversin-like protein, and include by way of specific example a protein comprising a PAS sequence and a Coversin-type protein sequence. PAS sequences are described e.g. in Schlapschy M, et al Protein Eng Des Sel. 2013 August; 26(8):489-501, and EP 08773567.6, with a PASylated Coversin molecule being described in Kuhn et al Bioconjugate Chem., 2016, 27 (10), pp 2359-2371. PASylation describes the genetic fusion of a protein with conformationally disordered polypeptide sequences composed of the amino acids Pro, Ala, and/or Ser. This is a technology developed by XL Protein (http://xl-protein.com/) and provides a simple way to attach a solvated random chain with large hydrodynamic volume to the protein to which it is fused. The polypeptide sequence adopts a bulky random coil structure. The size of the resulting fusion protein is thus much bigger than the protein to which it is fused. This has been shown to reduce clearance in biological systems. Appropriate PAS sequences are described in EP08773567.6, as well as the Schlapschy reference above. Any suitable PAS sequence may be used in the fusion protein. Examples include an amino acid sequence consisting of at least about 100 amino acid residues forming a random coil conformation and consisting of alanine, serine and proline residues (or consisting of proline and alanine residues). This may comprise a plurality of amino acid repeats, wherein said repeats consist of Ala, Ser, and Pro residues (or proline and alanine residues) and wherein no more than 6 consecutive amino acid residues are identical. Proline residues may constitute more than 4% and less than 40% of the amino acids of the sequence. The sequence may comprise an amino acid sequence selected from:

(SEQ ID NO: 15)
ASPAAPAPASPAAPAPSAPA,
(SEQ ID NO: 16)
AAPASPAPAAPSAPAPAAPS,
(SEQ ID NO: 17)
APSSPSPSAPSSPSPASPSS,
(SEQ ID NO: 18)
SAPSSPSPSAPSSPSPASPS,
(SEQ ID NO: 19)
SSPSAPSPSSPASPSPSSPA,
(SEQ ID NO: 20)
AASPAAPSAPPAAASPAAPSAPPA
and
(SEQ ID NO: 21)
ASAAAPAAASAAASAPSAAA

or circular permuted versions or multimers of these sequences as a whole or parts of these sequences. There may, for example be 5-40, 10-30, 15-25, 18-20 preferably 20-30 or 30 copies of one of the repeats present in the PAS sequence, i.e. one of SEQ ID NOs 15-21, preferably 15. Preferably the PAS sequence comprises or consists of 30 copies of SEQ ID NO:15. Preferably the PAS sequence is fused to the N terminus of the Coversin-type protein (directly or via a linker sequence), and in certain preferred embodiments the Coversin-type protein may comprise or consist of amino acids 19-168 of SEQ ID NO:2, or SEQ ID NO:34 or SEQ ID NO:35. e.g. the fusion protein comprises (a) a PAS sequence comprising or consisting of 30 copies of SEQ ID NO:15 and (b) (i) amino acids 19-168 of SEQ ID NO:2, (ii) SEQ ID NO:34, or (iii) SEQ ID NO:35, wherein (a) is fused to the N terminus of (b) directly or via a linker sequence).

Fusion proteins may additionally contain linker sequences (e.g. 1-50, 2-30, 3-20, 5-10 amino acids in length), such that the components are separated by this linker. In one embodiment the linker sequence can be a single alanine residue.

Compositions of fusion proteins comprising a PAS sequence can have increased viscosity compared to compositions comprising the non-PASylated version of the same protein. In some circumstances this increased viscosity can be disadvantageous. However, in the context of eye treatments in accordance with the present invention (e.g. topical eye treatments), increased viscosity may provide an advantage.

Coversin and functional equivalents thereof may be prepared in recombinant form by expression in a host cell. Such expression methods are well known to those of skill in the art and are described in detail by Sambrook et al (2000) and Fernandez & Hoeffler (1998). Recombinant forms of Coversin and functional equivalents thereof are preferably unglycosylated. Preferably the host cell is E. coli.

The Coversin-type protein is preferably in isolated form, e.g. separated from at least one component of the host cell and/or cell growth media in which it was expressed. In some embodiments, the Coversin-type protein is purified to at least 90%, 95% or 99% purity as determined, for example, by electrophoresis or chromatography. The Coversin-type proteins can also be prepared using conventional techniques of protein chemistry. For example, protein fragments may be prepared by chemical synthesis. Methods for the generation of fusion proteins are standard in the art and will be known to the skilled reader. For example, most general molecular biology, microbiology, recombinant DNA technology and immunological techniques can be found in Sambrook et al (2000) or Ausubel et al. (1991).

Preferably, the Coversin-type protein is not an antibody or a fusion protein.

It is further preferred that the Coversin-type protein is able to bind to both C5, in whatever polymorphic form, and LTB-4.

The Coversin-type protein may be used in combination with other pharmaceutical agents which are of use in treating disorders of the eye, such as an antihistamine, such as levocablastine, ketotifen or lodoxamide. Thus, where the Coversin-type protein is used in combination with one or more other treatment, this can be described as a Coversin-type protein (e.g. a protein comprising amino acids 19 to 168 of the amino acid sequence in FIG. 2 (SEQ ID NO: 2)) or a functional equivalent of this protein for use in a method of treating or preventing a cicatrising eye inflammatory disorder, in particular Sjögren's syndrome, mucous membrane pemphigoid or atopic keratoconjunctivitis with a second treatment, or as a second treatment for use in a method of treating or preventing a cicatrising eye inflammatory disorder, in particular Sjögren's syndrome, mucous membrane pemphigoid or atopic keratoconjunctivitis with a Coversin-type protein (e.g. a protein comprising amino acids 19 to 168 of the amino acid sequence in FIG. 2 (SEQ ID NO: 2)) or a functional equivalent of this protein.

It is envisaged that in all aspects of the present invention, the subject in need of treatment or prevention will predominantly be a human subject. However, all aspects of the invention can be used in connection with other subjects, such as mammalian subjects, in particular domestic mammals or farmed mammals.

The agent may be administered in a therapeutically or prophylactically effective amount. The term “therapeutically effective amount” refers to the amount of agent needed to treat the cicatrising eye inflammatory disorder. In this context, “treating” includes reducing the severity of the disorder.

The term “prophylactically effective amount” used herein refers to the amount of agent needed to prevent the cicatrising eye inflammatory disorder. In this context, “preventing” includes reducing the severity of the disorder, e.g. if the presence of the disorder is not detected before the administration of the agent is commenced.

The reduction or improvement is relative to the outcome without administration or the agent as described herein. The outcomes are assessed according to the standard criteria used to assess such patients. To the extent that this can be quantitated, there is a reduction or improvement of at least 10, 20, 30, 40, 50, 60, 70, 80, 90, 100% in the relative scoring criteria as discussed above.

In the drawings of this application:

FIG. 1 shows the classical and alternative complement pathways;

FIG. 2A shows the sequences for Coversin (SEQ ID NO:1 is the nucleotide sequence and SEQ ID NO:2 is the amino acid sequence);

FIG. 2B shows a number of Coversin mutants (SEQ ID Nos 4, 6, 8, 10, 12, 14, the DNA sequences encoding the corresponding fragments are SEQ ID NOS 3, 5, 7, 9, 11 and 13, respectively)

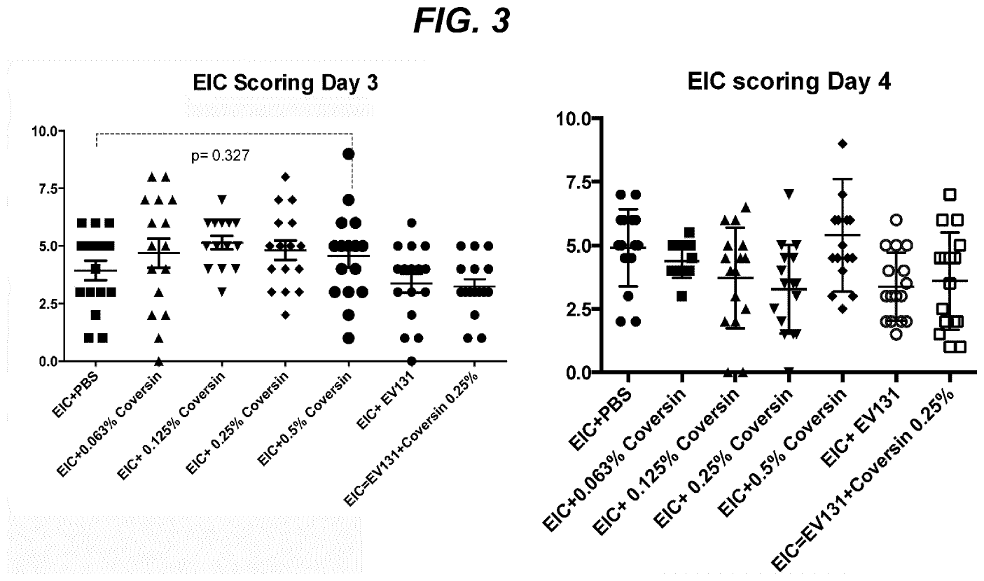

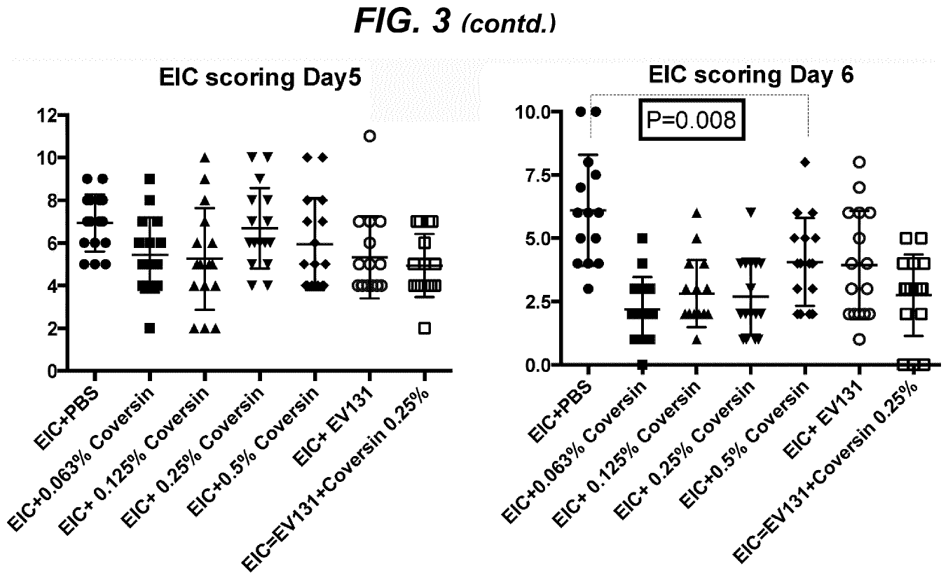

FIG. 3 shows EIC scoring for mice treated with ovalbumin and a variety of other agents, scored as a total from all eyes; and

FIG. 4 shows EIC scoring for mice treated with ovalbumin and a variety of other agents, scored as a mean of the two eyes in any given animal.

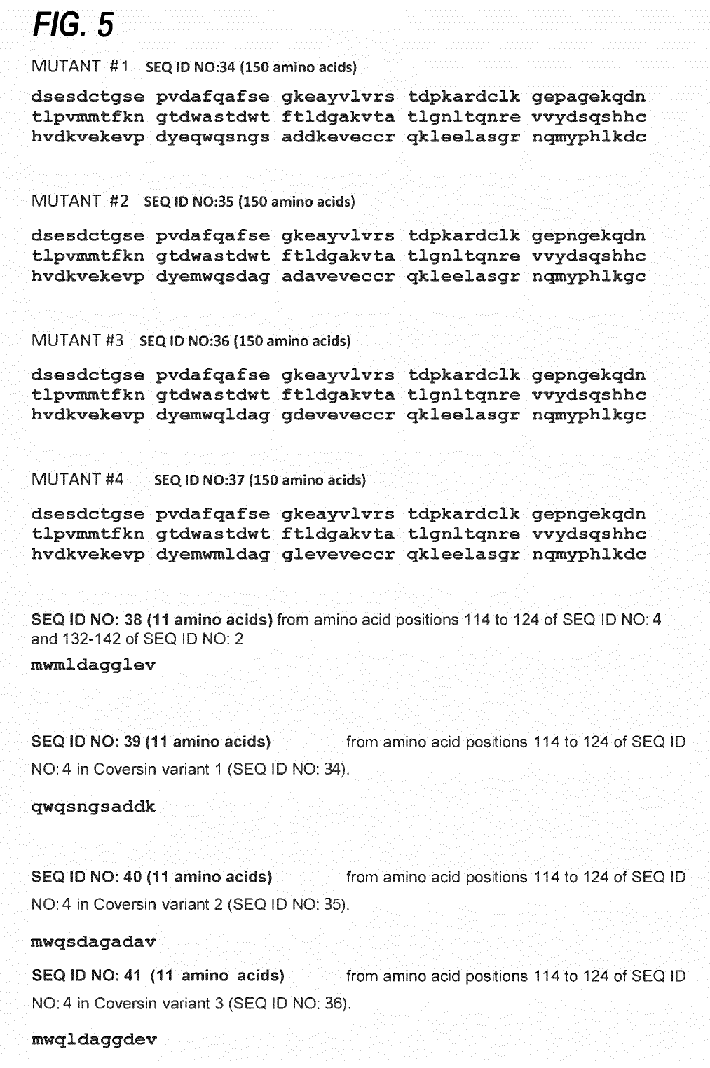

FIG. 5 shows the sequences of certain modified Coversin polypeptides with reduced or absent C5-binding activity but which retain LTB-4-binding ability.

The sequences as referred to in the present application are as follows:

SEQ ID NO:1 is the nucleotide sequence as shown in FIG. 2A. SEQ ID NO:2 is the amino acid sequence of Coversin as shown in FIG. 2A, including the 18 amino acid signal sequence that is absent in the mature protein,

SEQ ID NO:3 is the nucleotide sequence encoding SEQ ID NO:4

SEQ ID NO:4 is the mature Coversin amino acid sequence (amino acids 19-168 of SEQ ID NO:2)

SEQ ID NO:5 is the nucleotide sequence encoding SEQ ID NO:6

SEQ ID NO:6 is a 149 amino acid fragment of the mature Coversin amino acid sequence (amino acids 20-168 of SEQ ID NO:2).

SEQ ID NO:7 is the nucleotide sequence encoding SEQ ID NO:8

SEQ ID NO:8 is a 148 amino acid fragment of the mature Coversin amino acid sequence (amino acids 21-168 of SEQ ID NO:2).

SEQ ID NO:9 is the nucleotide sequence encoding SEQ ID NO:10

SEQ ID NO:10 is a 147 amino acid fragment of the mature Coversin amino acid sequence (amino acids 22-168 of SEQ ID NO:2).

SEQ ID NO:11 is the nucleotide sequence encoding SEQ ID NO:12

SEQ ID NO:12 is a 146 amino acid fragment of the mature Coversin amino acid sequence (amino acids 23-168 of SEQ ID NO:2).

SEQ ID NO:13 is the nucleotide sequence encoding SEQ ID NO:12

SEQ ID NO:14 is a 145 amino acid fragment of the mature Coversin amino acid sequence (amino acids 24-168 of SEQ ID NO:2).

SEQ ID Nos 15 to 21 are PAS sequences

SEQ ID Nos 22 to 33 are SEQ ID Nos 1-12 of WO 2015/185945

SEQ ID Nos 34 to 41 are sequences used to define certain modified Coversin polypeptides.

The present invention will now be described in more detail by way of example only in the following.

A mouse study was conducted according to the protocol described by Ahadome S. D. et. al. in XI Insight, 2016 Aug. 4; 1(12). pii: e87012. In this protocol, mice are first injected systemically with ovalbumin for 14 days and then, on Day 15 onwards, the eyes of the mice are challenged with topical ovalbumin. On Day 18 onwards, the eyes of the mice are treated with an optically-acceptable solution containing varying amounts of a Coversin-type protein or a control.

The mice were divided into 7 groups of animals. Group 1 (EIC+PBS) also received PBS in each eye. Group 2 (EIC+0.063% Coversin) also received PBS containing 0.063% (w/v) Coversin. This corresponds to 25.2 μg of Coversin at each application to each eye. Group 3 (EIC+0.125% Coversin) also received PBS containing 0.125% (w/v) Coversin. This corresponds to 50.4 μg of Coversin at each application to each eye. Group 4 (EIC+0.25% Coversin) also received PBS containing 0.25% (w/v) Coversin. This corresponds to 100.8 of Coversin at each application to each eye. Group 5 (EIC+0.5% Coversin) also received PBS containing 0.5% (w/v) Coversin. This corresponds to 201.6 μg of Coversin at each application to each eye. Group 6 (EIC+EV131) also received PBS containing EV131. Group 7 (EIC=EV131+Coversin 0.25%) also received PBS containing EV131 and 0.25% (w/v) Coversin.

All the mice were inspected by a trained investigator who graded the severity of the condition of each eye of each mouse and scored the eyes on a scale of 0 to 10, where 0 represents the absence of symptoms and 10 represents the most severe symptoms.

The results of the study are shown graphically in FIGS. 3 and 4. In FIG. 3, the score for each eye was recorded. In FIG. 4, the mean scores for a combination of the left and right eyes of each mouse was recorded. It can be seen from the Figures that Coversin reduced the scores for the mice, the best reductions being seen with lower doses after prolonged treatment (0.063% to 0.25% Coversin on Day 6).

This clearly demonstrates that topical treatment of eyes in sufferers from a cicatrising eye inflammatory disorder, in particular Sjögren's syndrome, mucuous membrane pemphigoid and atopic keratoconjunctivitis, with a Coversin-type protein will successfully reduce the symptoms.

The present invention has been described above by way of example only and the invention is not to be limited to the particular study referred to above. Rather the scope of the invention is defined by the appended claims.

1. A method for treating or preventing a cicatrising eye inflammatory disorder, in particular Sjögren's syndrome, mucous membrane pemphigoid or atopic keratoconjunctivitis, which comprises applying to a patient suffering from, or at risk of suffering from, said cicatrising eye inflammatory disorder a composition containing a protein comprising amino acids 19 to 168 of the amino acid sequence shown in FIG. 2 (SEQ ID NO:2) or a functional equivalent thereof.

2. The method of claim 1, wherein the composition is an optically-acceptable composition and the composition is applied topically to the eye of the patient.

3. The method of claim 1, wherein the method further includes treating the eye with a conventional antihistamine.

4-6. (canceled)

7. The method of claim 1, wherein the patient is a human.

8. The method of claim 1, wherein the functional equivalent of the protein comprising amino acids 19 to 168 of the amino acid sequence shown in FIG. 2 (SEQ ID NO:2) is a protein comprising a sequence having at least 90% sequence identity to the sequence of amino acids 19 to 168 of SEQ ID NO: 2, and said protein binds C5 to prevent the cleavage of complement C5 by convertase into complement C5a and complement C5b and/or binds LTB4.

9. The method of claim 1, wherein the functional equivalent of the protein comprising amino acids 19 to 168 of the amino acid sequence shown in FIG. 2 (SEQ ID NO:2) is a protein comprising a sequence having at least 95% sequence identity to the sequence of amino acids 19 to 168 of SEQ ID NO: 2, and said protein binds C5 to prevent the cleavage of complement C5 by convertase into complement C5a and complement C5b and/or binds LTB4.

10. The method of claim 1, wherein the functional equivalent of the protein comprising amino acids 19 to 168 of the amino acid sequence shown in FIG. 2 (SEQ ID NO:2) is a protein comprising the sequence of amino acids 19 to 168 of SEQ ID NO: 2, in which up to 10 amino acid substitutions, insertions or deletions have been made, and said protein binds C5 to prevent the cleavage of complement C5 by convertase into complement C5a and complement C5b and/or binds LTB4.

11. The method of claim 1, wherein the functional equivalent of the protein comprising amino acids 19 to 168 of the amino acid sequence shown in FIG. 2 (SEQ ID NO:2) is a fragment of the protein as defined in any of claims 8 to 10, and said protein binds C5 to prevent the cleavage of complement C5 by convertase into complement C5a and complement C5b and/or binds LTB4.

12. The method of claim 1, wherein the functional equivalent has the ability to bind to both C5 and LTB-4.

13. The method of claim 1, wherein the functional equivalent of the protein comprising amino acids 19 to 168 of the amino acid sequence shown in FIG. 2 (SEQ ID NO:2) is a RaCI protein or an antibody to C5.

14. The method of claim 1, wherein the functional equivalent of the protein comprising amino acids 19 to 168 of the amino acid sequence shown in FIG. 2 (SEQ ID NO:2) is a fusion protein comprising (a) a sequence having at least 90% sequence identity to the sequence of amino acids 19 to 168 of SEQ ID NO: 2, and (b) a second sequence and said protein binds C5 to prevent the cleavage of complement C5 by convertase into complement C5a and complement C5b and/or binds LTB4.

15. The method of claim 14 wherein said second sequence is a PAS sequence.


SEQUENCE LISTING (41 sequences)

SEQ ID NO: 1 - DNA, 507 bp, Ornithodoros moubata
atgctggttttggtgaccctgattttctccttttctgcgaacatcgcatatgctgacagcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 2 - protein, 168 aa, Ornithodoros moubata
MLVLVTLIFSFSANIAYADSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 3 - DNA, 453 bp, Ornithodoros moubata
gacagcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 4 - protein, 150 aa, Ornithodoros moubata
DSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 5 - DNA, 450 bp, Ornithodoros moubata
agcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 6 - protein, 149 aa, Ornithodoros moubata
SESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 7 - DNA, 447 bp, Ornithodoros moubata
gaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 8 - protein, 148 aa, Ornithodoros moubata
ESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 9 - DNA, 444 bp, Ornithodoros moubata
agcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 10 - protein, 147 aa, Ornithodoros moubata
SDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 11 - DNA, 441 bp, Ornithodoros moubata
gactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 12 - protein, 146 aa, Ornithodoros moubata
DCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 13 - DNA, 438 bp, Ornithodoros moubata
tgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 14 - protein, 145 aa, Ornithodoros moubata
CTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 15 - protein, 20 aa, Artificial Sequence
  PAS sequence
ASPAAPAPASPAAPAPSAPA

SEQ ID NO: 16 - protein, 20 aa, Artificial Sequence
  PAS sequence
AAPASPAPAAPSAPAPAAPS

SEQ ID NO: 17 - protein, 20 aa, Artificial Sequence
  PAS sequence
APSSPSPSAPSSPSPASPSS

SEQ ID NO: 18 - protein, 20 aa, Artificial Sequence
  PAS sequence
SAPSSPSPSAPSSPSPASPS

SEQ ID NO: 19 - protein, 20 aa, Artificial Sequence
  PAS sequence
SSPSAPSPSSPASPSPSSPA

SEQ ID NO: 20 - protein, 24 aa, Artificial Sequence
  PAS sequence
AASPAAPSAPPAAASPAAPSAPPA

SEQ ID NO: 21 - protein, 20 aa, Artificial Sequence
  PAS sequence
ASAAAPAAASAAASAPSAAA

SEQ ID NO: 22 - protein, 78 aa, Rhipicephalus appendiculatus
EEVKTTPIPNHQCVNATCERKLDALGNAVITKCPQGCLCVVRGASNIVPANGTCFQLATTKPPMAPGDNKDNKEEESN

SEQ ID NO: 23 - protein, 74 aa, Rhipicephalus appendiculatus
TAEATLSINGGDMCIEKTCNRSIDAAGKKVIAGCPGGCLCVFNVSDVTYPANGTCYQLATTTTNRPGAVMERER

SEQ ID NO: 24 - protein, 78 aa, Rhipicephalus appendiculatus
SGESQSIQRKGQCEEVICHRKLNHLGERVTSGCPTGCLCVIREPDNVDNANGTCYALMSSTTTTTTTPDGTTTSEEEE

SEQ ID NO: 25 - protein, 72 aa, Rhipicephalus appendiculatus
QEPTTPLKAASQCSNVKCRRRFDHLGNSVTEGCPSGCLCVYQATGYNQEANGTCYELMKTSTTTTTEGTPAQ

SEQ ID NO: 26 - protein, 75 aa, Rhipicephalus appendiculatus
SGESQSIQRKGQCEEVTCHRTLNHLGVAVTSGCPSGCLCVISAPDSAVNVNGTCYQLMGSTSTTTSSTPSSEDQE

SEQ ID NO: 27 - protein, 77 aa, Rhipicephalus appendiculatus
EEANTTPISVKDQCANVTCRRTVDNRGKRHIDGCPPGCLCVLKGPDSKDNLDGTCYLLATTPKSTTTSTEQSFNMEE

SEQ ID NO: 28 - protein, 78 aa, Rhipicephalus appendiculatus
SGESQSIQRNGRCEEVTCQRKPNHLGVAVTSGCPPGCLCVIQAPDNAVNANGTRYELMTTTTTKTTTTSGTPSSEDPE

SEQ ID NO: 29 - protein, 77 aa, Rhipicephalus appendiculatus
EEANTTPISVKDQCANVTCRRTVDNRGKRHIDGYPPGCLCVLKGPDSKDNLDGTCYLLTTTPKPTTTSTEQSFNMEE

SEQ ID NO: 30 - protein, 98 aa, Rhipicephalus appendiculatus
MNAMLVLFIASALFISEHNTEEVKTTPIPNHQCVNATCERKLDALGNAVITKCPQGCLCVVRGASNIVPANGTCFQLATTKPPMAPGDNKDNKEEESN

SEQ ID NO: 31 - protein, 104 aa, Rhipicephalus appendiculatus
MKLCILLAVVAFVGLSLGHHHHHHAGEEVKTTPIPNHQCVNATCERKLDALGNAVITKCPQGCLCVVRGASNIVPANGTCFQLATTKPPMAPGDNKDNKEEESN

SEQ ID NO: 32 - protein, 86 aa, Rhipicephalus appendiculatus
HHHHHHAGEEVKTTPIPNHQCVNATCERKLDALGNAVITKCPQGCLCVVRGASNIVPANGTCFQLATTKPPMAPGDNKDNKEEESN

SEQ ID NO: 33 - protein, 42 aa, Rhipicephalus appendiculatus
CVNATCERKLDALGNAVITKCPQGCLCVVRGASNIVPANGTC

SEQ ID NO: 34 - protein, 150 aa, Artificial Sequence
  Coversin variant 1
DSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEQWQSNGSADDKEVECCRQKLEELASGRNQMYPHLKDC

SEQ ID NO: 35 - protein, 150 aa, Artificial Sequence
  Coversin variant 2
DSESDCTGSEPVDAFQAFSEGKEAYVLVRSTDPKARDCLKGEPNGEKQDNTLPVMMTFKNGTDWASTDWTFTLDGAKVTATLGNLTQNREVVYDSQSHHCHVDKVEKEVPDYEMWQSDAGADAVEVECCRQKLEELASGRNQMYPHLKGC

SEQ ID NO: 36 - protein, 150 aa, Artificial Sequence
  Coversin variant 3
DSESDCTGSEPVDAFQAFSEGKEAYVLVRSTDPKARDCLKGEPNGEKQDNTLPVMMTFKNGTDWASTDWTFTLDGAKVTATLGNLTQNREVVYDSQSHHCHVDKVEKEVPDYEMWQLDAGGDEVEVECCRQKLEELASGRNQMYPHLKGC

SEQ ID NO: 37 - protein, 150 aa, Artificial Sequence
  Coversin variant 4
DSESDCTGSEPVDAFQAFSEGKEAYVLVRSTDPKARDCLKGEPNGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 38 - protein, 11 aa, Artificial Sequence
  Loop sequence
MWMLDAGGLEV

SEQ ID NO: 39 - protein, 11 aa, Artificial Sequence
  Loop sequence of Coversin variant 1
QWQSNGSADDK

SEQ ID NO: 40 - protein, 11 aa, Artificial Sequence
  Loop sequence of Coversin variant 2
MWQSDAGADAV

SEQ ID NO: 41 - protein, 11 aa, Artificial Sequence
  Loop sequence of Coversin variant 3
MWQLDAGGDEV